(19) United States
(12) Patent Application Publication
(10) Pub. No.: US 2026/0166148 A1
(43) Pub. Date: Jun. 18, 2026

(54) MAGNETOSTRICTIVE PIEZOELECTRIC NANOASSEMBLY AS CANCER CHEMOTHERAPEUTIC

(71) Applicant: GRANT DEMARTINO INDUSTRIES LLC, Yonkers, NY (US)

(72) Inventors: Duane GRANT, Bronx, NY (US); Amanda GREY-RODRIGUEZ, Bronx, NY (US)

(21) Appl. No.: 18/709,655

(22) PCT Filed: Oct. 25, 2022
(86) PCT No.: PCT/IB2022/060250

§ 371 Date: May 13, 2024

Related U.S. Application Data
(60) Provisional application No. 63/263,067, filed on Oct. 26, 2021.
(60) Provisional application No. 63/309,278, filed on Feb. 11, 2022.
(60) Provisional application No. 63/371,250, filed on Aug. 12, 2022.

(51) Int. Cl. A61K 41/00; A61K 9/51

(52) U.S. Cl. CPC A61K 41/0052; A61K 9/5115; A61K 9/5123; A61K 9/5138; A61K 9/5146; A61K 9/5161

(57) ABSTRACT

A nanoparticle assembly is disclosed for the treatment and visualization of cancers. The assembly includes a core and two surrounding copolymer layers. The surrounding layers include hydrogel polymers, dextran-iron oxide nanoparticles, and quantum dots having a tail with a photosensitizer and an aptamer targeting cancer cells. Exposure of the assembly to an AC magnetic field creates heat and vibration from magnetostrictive nanobeads in the core. Piezoelectric elements generate electric charges from the vibration that activate fluorescence in the quantum dots, which in turn activate the photosensitizer to generate reactive oxygen species that induce apoptosis in cancer cells. Alternative anticancer mechanisms include thermolytic activation of oxygen independent cytotoxic free radicals from heat caused by magnetostrictive vibration. Thermolysis can also release immunological factors embedded in hydrogel polymers. 2-¹⁸fluoro-2-deoxyglucose (¹⁸F-FDG) may be incorporated as a diagnostic tool that can visualize cancer sites with PET-CT. Methods of preparing the nanoparticle assembly are disclosed.

### FIELD OF THE INVENTION

A nanoassembly and method for killing cancer cells is disclosed relying on magnetostrictive vibration that generates piezoelectric charges and heat. Piezoelectric charges activate quantum dots that activate photosensitizers to generate cytotoxic reactive oxygen species at the site of a tumor. The heat causes thermolytic generation of oxygen independent cytotoxic free radicals and thermolytic release of anticancer immunological factors. Additionally, ¹⁸Fluoro-2-deoxyglucose (¹⁸F-FDG) that may be incorporated into tumor targeting features useful as a diagnostic tool that can visualize cancer sites with PET-CT. Methods of preparing the nanoassembly are disclosed.

### BACKGROUND

Cancer (generically) is a disease in which some of the body's cells grow uncontrollably and spread (metastasize) to other parts of the body. Cancers can start almost anywhere in the body. Cancers occur when the normally orderly and highly regulated cell division and regeneration mechanisms in the body breaks down, and abnormal or damaged cells grow and multiply inappropriately. This is also termed a neoplasm. In some cases, neoplasms may form solid tumors. Solid tumors can be further characterized as malignant or benign. Malignant tumors are cancerous and are spreadable to other parts of the body and are characterized by anaplasia, invasiveness, and metastasis. Benign tumors do not spread, although they can become large and dangerous if untreated. Cancers may also be non-solid, for example cancers of the blood or lymph system.

Cancers are classified by the type of cell that the tumor cells resemble and is therefore presumed to be the origin of the tumor. These types include carcinomas, sarcomas, lymphomas and leukemia, germ cell tumors and blastomas.

The growth and proliferation of all forms of cancer remains a serious medical issue despite the progressive development of different methods of treatment. These treatments have certain advantages, but also have unique disadvantages ranging from inadequate penetration depth (i.e., photodynamic therapy) to extensive adverse physiological effects on normal, healthy tissue (i.e., chemotherapy & radiation therapy), and lack of effectiveness.

In general, medical treatments for tumors involve surgical removal of tumors or any of various methods of killing cancer cells, such as phototherapy, radiotherapy, and chemotherapy, typically with drugs that kill cells or prevent cellular division.

With chemotherapy in particular, methods of chemically targeting malignant cells can be important. Highly toxic drugs can be linked to a moiety that ideally would target and attach to malignant cells and only release their “warhead” on cells that are actually malignant, and not on healthy cells which can also be killed by the warhead. Moreover, an additional complication is that complex molecules and nanoparticles can be difficult to deliver to the cytoplasm of cells. [1]

There remains an ongoing need for new cancer therapies. Existing treatments often fail to effectively treat tumors and existing treatments often have severe toxicity and side effects. Accordingly, improved therapies are being sought.

### SUMMARY OF THE INVENTION

This invention relies in part on photodynamic therapy as a chemotherapeutic modality. A nanoassembly is provided having aptamers to target malignant cells, and the nanoassembly is activated by alternating current (AC) magnetic fields that cause magnetostrictive elements to vibrate and generate electric charges and/or heat. The electric charges activate quantum dots that provide light energy to activate photosensitive moieties that generate reactive oxygen species. Alternatively, heat generated from vibrational energy generates cytotoxic free radicals or release immunological factors from the nanoassembly. This cascade of events leads to apoptosis and cell death of malignant cells. Additionally, 2-¹⁸fluoro-2-deoxyglucose (FDG) may be incorporated into the nanoassembly to allow visualization of tumor sites by FDG positron emission tomography (PET).

In an embodiment, a nanoparticle assembly is provided for the treatment and visualization of a cancer, having an assembly core (7) that includes a plurality of nanobeads (7a), where each nanobead includes one or more materials that combine to result in a magnetoelectric effect for the core 7. In an embodiment, nanobeads 7a have a nested core-shell configuration wherein the core comprises CoFe₂O₄ (CFO) and the shell comprises BaTiO₃ (BTO), and wherein the nanobead is magnetoelectric.

The assembly core may be bounded by an intermediate copolymer layer hydrogel 9 comprising poly(3,4-ethylenedioxythiophene) polystyrene sulfonate treated with sorbitol (D-Sorbitol-PEDOT: PSS) (4), a biocompatible polymer filler (5) that adjusts the lower critical solution temperature (LCST) of N-isopropyl acrylamide (6). In an embodiment, the biocompatible polymer filler is glycidyl methacrylate/glycidyl methacrylate hyaluronic acid (HA-GMA/GMHA) (5). Also included may be an azobis compound (3) that generates free radicals thermolytically, and aminooxyacetic group-conjugated dextran-iron oxide hybrid nanoparticles (2a), and wherein the intermediate layer optionally contains one or more immunological factors (8), and the intermediate layer (9) is electroconductive. The assembly may further include an outer layer (10) including electroluminescent InP/ZnS or CdSe/ZnS quantum dots (1a) with emission peaks at one or more of 683 763, and 785 nm, and wherein a tail moiety (1d) is conjugated to each quantum dot, wherein the tail has one or more photosensitizer linkages and one or more aptamers wherein the aptamers bind to a receptor overexpressed on cancer cells. The outer layer (10) may include iron oxide hybrid nanoparticles (2) having an iron oxide nanoparticle core with a tail of one or more aminooxyacetate linkers (2b) and optionally one or more 2-¹⁸Fluoro-2-deoxyglucose (¹⁸F-FDG) subunits (2c); wherein the nanoparticles (2) may further include a polyethylene glycol (2d) moiety conjugated thereto.

In an embodiment, the photosensitizer is selected from a hematoporphyrin, a hematophorphyrin ester, a dihematophorphyrin ester, a boron dipyrromethene (BODIPY) and a ADPM06 class of drugs, or azadipyrromethene/ADPM06, padeliporfin, photofrin/porfimer, and tin ethyl etiopurpurin.

In an embodiment, the immunological factors are a stimulator of interferon genes (STING) agonist or an ectonucleotide pyrophosphatase/phosphodiesterase 1 (ENPP1) inhibitor and are selected from one or more of ADU-S100/MIW815, SR-8541A, SR-8314-ENPP1 Inhibitor, MK-1454, SB11285, and BI-STING (BI 1387446).

In an embodiment, the immunological factors stimulate macrophages, CD4, or CD8 cells or a combination thereof and are selected from N-formylmethionine-leucyl-phenylalanine (fMLF) and neoleukin-2/15, and NL-201.

In an embodiment, the azobis compound is selected from 2,2′-Azobis(2-(2-imidazolin-2-yl) propane) dihydrochloride (AIPH); dimethyl 2,2′-azobis (2-methylpropionate) (AIBME); and 2,2′-Azobis(2-amidinopropane) dihydrochloride (AAPH).

In an embodiment, the quantum dots are InP/ZnS quantum dots.

In an embodiment, a nanoparticle assembly is provided for the treatment and visualization of a cancer with a photosensitizer. The assembly may have core (7) including a plurality of nanobeads (7a), where each nanobead comprises one or more materials that combine to result in a magnetoelectric effect for the core 7. In an embodiment, nanobeads 7a have a nested core-shell configuration wherein the core comprises CoFe₂O₄ (CFO) and the shell comprises BaTiO₃ (BTO), and wherein the nanobead is magnetoelectric. The assembly core may be bounded by an intermediate copolymer hydrogel layer (9) comprising D-Sorbitol-PEDOT: PSS (4), glycidyl methacrylate modified hyaluronic acid (5), and N-isopropyl acrylamide (6), embedded with 2,2′-azobis (2-(2-imidazolin-2-yl) propane) dihydrochloride (3), and aminooxyacetic group-conjugated dextran-iron oxide hybrid nanoparticles (2a), and wherein the intermediate layer (9) is electroconductive. The assembly may also include an outer layer (10) comprising electroluminescent InP/ZnS or CdSe/ZnS quantum dots (1a) with emission peaks at one or more of 683 763, and 785 nm, and wherein a tail moiety (1d) is conjugated to each quantum dot, wherein the tail may have one or more photosensitizer linkages and one or more aptamers wherein the aptamers bind to a receptor overexpressed on cancer cells. The outer layer (10) may also include iron oxide hybrid nanoparticles (2) with an iron oxide nanoparticle core with a tail having one or more aminooxyacetate linkers (2b) and optionally one or more 2-¹⁸Fluoro-2-deoxyglucose (¹⁸F-FDG) subunits (2c); wherein the nanoparticles (2) may further include a polyethylene glycol (2d) moiety conjugated thereto.

In an embodiment, a nanoparticle assembly is provided for the treatment and/or visualization of a cancer by thermolytic generation of cytotoxic free radicals at the site of a tumor or cancerous cells. The nanoparticle assembly may have core (7) including a plurality of nanobeads (7a), where each nanobead comprises one or more materials that combine to result in a magnetoelectric effect for the core 7. In an embodiment, nanobeads 7a have a nested core-shell configuration wherein the core comprises CoFe₂O₄ (CFO) and the shell comprises BaTiO₃ (BTO), and wherein the nanobead is magnetoelectric. The assembly core may be bounded by an intermediate copolymer hydrogel layer (9) comprising D-Sorbitol-PEDOT: PSS (4), glycidyl methacrylate modified hyaluronic acid (5), N-isopropyl acrylamide (6), an azobis compound (3) that generates free radicals thermolytically, and aminooxyacetic group-conjugated dextran-iron oxide hybrid nanoparticles (2a). The assembly may also include an outer layer (10) comprising iron oxide hybrid nanoparticles (2) comprising an iron oxide nanoparticle core with a tail comprising one or more aminooxyacetate linkers (2b) and optionally one or more 2-¹⁸Fluoro-2-deoxyglucose (¹⁸F-FDG) subunits (2c); wherein the nanoparticles (2) further comprise a polyethylene glycol (2d) moiety and an aptamer conjugated thereto.

In an embodiment, a nanoparticle assembly is provided for the treatment and/or visualization of a cancer by the release of immunological factors at the site of a tumor or cancerous cells. The nanoparticle assembly may have core (7) including a plurality of nanobeads (7a), where each nanobead comprises one or more materials that combine to result in a magnetoelectric effect for the core 7. In an embodiment, nanobeads 7a have a nested core-shell configuration wherein the core comprises CoFe₂O₄ (CFO) and the shell comprises BaTiO₃ (BTO), and wherein the nanobead is magnetoelectric. The assembly core may be bounded by an intermediate copolymer hydrogel layer (9) comprising D-Sorbitol-PEDOT: PSS (4), glycidyl methacrylate modified hyaluronic acid (5), and N-isopropyl acrylamide (6), and aminooxyacetic group-conjugated dextran-iron oxide hybrid nanoparticles (2a), and wherein the intermediate layer contains one or more immunological factors (8). The intermediate layer may be electroconductive. The assembly may further include an outer layer (10) comprising aptamers linked to the iron oxide nanoparticle bind to a receptor overexpressed on cancer cells. The outer layer (10) may further include iron oxide hybrid nanoparticles (2) with an iron oxide nanoparticle core with a tail having one or more aminooxyacetate linkers (2b) and optionally one or more 2-¹⁸Fluoro-2-deoxyglucose (¹⁸F-FDG) subunits (2c); wherein the nanoparticles (2) further comprise a polyethylene glycol (2d) moiety and an aptamer conjugated thereto.

In an embodiment, a nanoparticle assembly for the visualization of a cancer, is provided, wherein the assembly comprises a copolymer hydrogel layer (9) comprising D-Sorbitol-PEDOT: PSS (4), a biocompatible polymer filler (5) that adjusts the LOST of N-isopropyl acrylamide (6), and aminooxyacetic group-conjugated dextran-iron oxide hybrid nanoparticles (2). The iron oxide hybrid nanoparticles (2) may include an iron oxide nanoparticle core with a tail comprising one or more aminooxyacetate linkers (2b) and one or more 2-¹⁸Fluoro-2-deoxyglucose (¹⁸F-FDG) subunits (2c) conjugated to the nanoparticle core. The nanoparticles (2) may further comprise a polyethylene glycol (2d) moiety and an aptamer conjugated thereto wherein the aptamer binds to a receptor overexpressed on cancer cells.

In an embodiment, a method of killing cancer cells in a patient suffering from a cancerous tumor is provided. The method includes the administration of a nanoassembly as described above to a patient suffering from a cancer or malignant tumor, waiting a period of time following administration to allow the assemblies to enter into or attach to the surface of cancer cells, and subjecting an area affected by cancer to an alternating current (AC) magnetic field. The AC field causes magnetostriction in nanobeads 7a, for example from CoFe₂O₄ cores of a CoFe₂O₄/BaTiO₃ piezoelectric nanobeads (7a), which induces mechanical strain on the BaTiO₃ shell of the 7a nanobeads, which generates an electric polarization and charge separation, i.e., from a piezoelectric effect. The electric charge is transmitted through the electroconductive intermediate hydrogel layer 9 containing D-Sorbitol-PEDOT: PSS (4) copolymer to the quantum dots (1a). The quantum dots are activated by the electric charge and emit light at one or more wavelengths of 683 763, and 785 nm. The light emission from the quantum dots activates the photosensitizer moieties (1b) conjugated to the quantum dots, and the activated photosensitizers generate reactive oxygen species (ROS's) within or on the surface of cancer cells, thereby inducing cellular damage and apoptosis in cancer cells.

In an embodiment, a method of killing cancer cells in a patient suffering from a cancerous tumor is provided, wherein the method includes administration of the nanoassembly as described above to a patient suffering from a cancer or malignant tumor, waiting a period of time following administration to allow the assemblies to enter into or attach to the surface of cancer cells, and subjecting an area affected by cancer to an alternating current (AC) magnetic field. The AC magnetic field causes induces magnetostriction in the nanobeads (7a), and magnetic induction generates heat causing the polymer material (4) to deform and warm up, thereby generating oxygen independent free radicals from an azobis free radical precursor (3). The free radicals induce cellular damage and apoptosis in cancer cells.

In an embodiment, a method of killing cancer cells in a patient suffering from a cancerous tumor is provided, wherein the method includes administration of the nanoassembly as described above to a patient suffering from a cancer or malignant tumor, waiting a period of time following administration to allow the assemblies to enter into or attach to the surface of cancer cells, and subjecting an area affected by cancer to an alternating current (AC) magnetic field. The AC magnetic field induces magnetostriction in the nanobeads (7a), and wherein magnetic induction generates heat causing the polymer material (4) to deform and warm up, thereby releasing immunological factors into the local environment of malignant cells.

In an embodiment, the immunological factors are a stimulator of interferon genes (STING) agonist or an ectonucleotide pyrophosphatase/phosphodiesterase 1 (ENPP1) inhibitor and may include one or more of ADU-S100/MIW815, SR-8541A, SR-8314-ENPP1 Inhibitor, MK-1454, SB11285, and BI-STING (BI 1387446).

In an embodiment, the immunological factors stimulate macrophages, CD4, or CD8 cells or a combination thereof and are selected from N-formylmethionine-leucyl-phenylalanine (fMLF) and neoleukin-2/15, and NL-201.

In an embodiment, the nanoassembly is guided to the site of a tumor with a DC magnetic field with or without computed tomographic scanning visualization.

### DESCRIPTION OF THE DRAWINGS

FIG. 1 is a cross section of a nanoparticle assembly 100 according to the instant invention, showing the domains 7, 9, and 10, and representative constituent sub-parts.

FIG. 2 is a bisected perspective view of a nanoparticle assembly 100 according to the instant invention, showing the domains 7, 9, and 10, and representative constituent sub-parts.

FIG. 3 shows details of the quantum dots 1, 1a, 1b, 1c, and 1d of this invention and parts conjugated thereto.

FIG. 4 shows details of the dextran-iron oxide nanoparticles 2, 2a, 2b, 2c, and 2d of this invention.

FIG. 5 shows 2,2′-Azobis(2-(2-imidazolin-2-yl) propane) dihydrochloride (AIPH), marked as 3.

FIG. 6 shows various components of the three component copolymer surface (4, 4a, 4b, 4c).

FIG. 7 shows HA-GMA 5 and constituent structures.

FIG. 8 shows schematic representations of a piezoelectric nanobead 7 and 7a.

FIG. 9 shows N-isopropyl acrylamide (NIPAM) structure 6.

FIG. 10 illustrates some immunogenic factors 8, 8a, and 8b disclosed in this invention.

FIG. 11 is a flow chart showing a sequence of events leading to cell death and other functions of the instant invention.

FIG. 12 is a flow chart of methods of assembly of the inventive nanoassemblies.

### DETAILED DESCRIPTION

In an embodiment, this invention provides a nanoassembly-based three-component theranostic treatment (specifically for cancers) with photosensitive drug particulates and a built-in light source via a hybrid nanoparticle with a net magnetofluorescent character. Upon administration, the nanoassembly (also termed a nanoparticle) can be guided to a location using a DC magnetic field gradient, by aptamers with an affinity for tumor cells, or by other means. The localization can be visualized using PET-CT from an integrated ¹⁸F isotope. The nanoassemblies are then activated via an AC magnetic field. The AC magnetic field causes a dual effect. First, there is a cascade from piezoelectric behavior that activates near infrared emission from quantum dots in the nanoassembly, allowing for a photodynamic reaction via integrated photosensitizer compounds that generates reactive oxygen species that are toxic to cells. Second, there is a heating effect from vibrational energy from iron oxide nanocrystals in a hydrogel matrix. This heating causes the hydrogel to shrink and thermally generates oxygen independent cytotoxic free radical compounds in the target area. This pathway is particularly effective against hypoxic tumors. Additionally, the heating effect may release immunological factors intended to activate intrinsic immune responses that will kill cancer cells. Following administration, the magnetic components can be removed at will by external magnetic guidance, while the remaining biodegradable components can be naturally filtered by the body by multiple pathways.

Although this invention is described in the foregoing paragraph as providing three independent methods of killing cancer cells, alternative embodiments may provide a nanoparticle-based treatment using one of these methods or any combination of two of these methods. The diagnostic features such as an ¹⁸F radioisotope discussed herein may or may not be combined with any of these combinations.

The term “theranostic” means that the inventive nanoassemblies are useful for both therapeutic and diagnostic purposes. The inventive nanoassemblies including targeting moieties designed to specifically bind to cancer cells and may include an ¹⁸F radioisotope for medical imaging with positron emission tomography (FDG-PET) of a tumor site.

The term “magnetofluorescent” means that a fluorescent effect in quantum dots is activated by an AC magnetic field. As used herein, the terms “cancerous,” “tumorous,” and “malignant” are synonymous.

In an embodiment, a nanoparticle assembly for the treatment and visualization of a cancer is provided. The nanoassembly may include an assembly core (7) having a plurality of nanobeads (7a), where each nanobead may have a one or more materials that combine to result in a magnetoelectric effect for the core 7. For example, the nanobeads may have a nested core-shell configuration wherein the core comprises CoFe₂O₄ and the shell comprises BaTiO₃, and wherein the nanobeads are magnetoelectric.

The assembly core may be bounded by an intermediate hydrogel layer (9) that may be a copolymer of sorbitol treated poly(3,4-ethylenedioxythiophene) polystyrene sulfonate (D-Sorbitol-PEDOT: PSS) (4). Other components of the hydrogel layer may include a biocompatible polymer filler (5) that adjusts the lower critical solution temperature (LCST) of N-isopropyl acrylamide (6), an azobis compound (3) that generates free radicals thermolytically, and aminooxyacetic group-conjugated dextran-iron oxide hybrid nanoparticles (2a). The intermediate hydrogel layer optionally contains one or more immunological factors (8), and the intermediate layer (9) may be electroconductive.

The assembly may further include an outer layer (10) having a hydrophilic surface conducive to interacting with tissues in vivo. Outer layer 10 may include electroluminescent InP/ZnS or CdSe/ZnS quantum dots (1a) with emission peaks at one or more of 683 763, and 785 nm, and wherein a tail moiety (1d) is conjugated to each quantum dot. The tail may include one or more photosensitizer linkages and one or more aptamers wherein the aptamers bind to a receptor overexpressed on cancer cells. The outer layer (10) may also include iron oxide hybrid nanoparticles (IONP's, 2) comprising an iron oxide nanoparticle core with a tail that may include one or more aminooxyacetate linkers (2b) and optionally one or more 2-¹⁸Fluoro-2-deoxyglucose (¹⁸F-FDG) subunits (2c). The nanoparticles (2) may also include a polyethylene glycol (2d) moiety conjugated thereto.

### Nanoparticle Structure

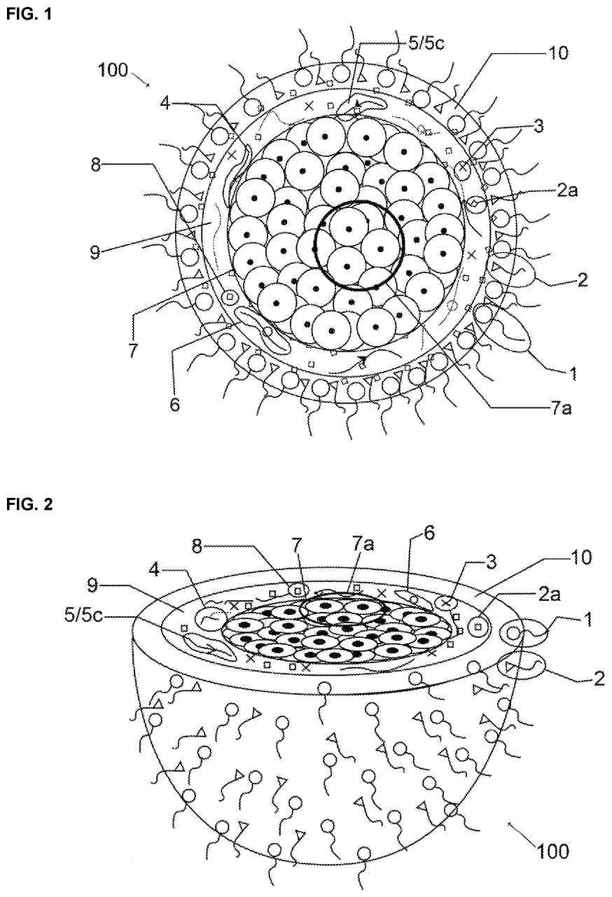

In an embodiment, a hybrid nanoparticle assembly 100 (FIGS. 1 and 2) is provided. The assembly 100 may have a multilayer nested core-shell configuration with a core region 7, an intermediate layer region 9, and an outer layer 10. Nanoassembly 100 may be a colloid in water with one or more additional excipients for use as a therapeutic as disclosed here.

In an embodiment, core region 7 comprises a plurality of nanobeads (7a, FIG. 8) composed of one or more materials that combine to result in a magnetoelectric effect for the core 7. In an embodiment, the nanobeads may be CoFe₂O₄—BaTiO₃ core-shell magnetoelectric nanobeads with CoFe₂O₄ (cobalt ferrite, CFO) as the nanobead core material and BaTiO₃ (barium titanate, BTO) as the nanobead shell material. The CFO material is magnetostrictive, meaning that it can change shape and dimensions under the influence of an AC magnetic field. The 7a nanobeads may further have piezoelectric characteristics. Piezoelectric devices generate an electric polarization and charge separation from mechanical stress. CFO/BTO nanobeads have such a piezoelectric features, where the piezoelectric effect is caused from magnetostriction of the CFO core induced by an AC magnetic field, which causes the CFO core to vibrate. The vibration creates pressure on the BTO shell which generates an electric polarization and charge separation. This creates a piezoelectric charge which is used to activate quantum dots, as explained below. In an embodiment, the 7a nanobeads are about 5-10 nm in diameter.

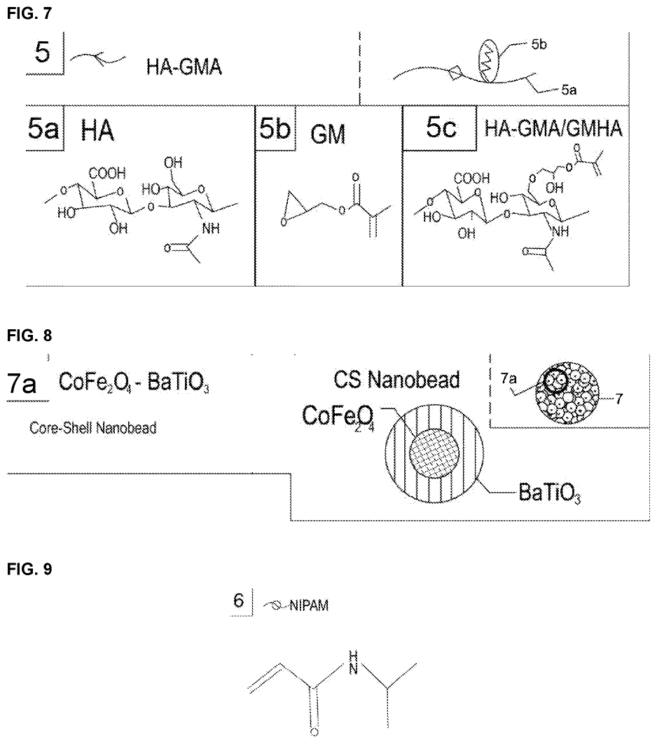

The nanobeads in the assembly core region 7 are bounded by a heterogenous hydrogel layer (9) that may include a three component copolymer surface (4, 5, 6) comprising sorbitol-treated poly(3,4-ethylenedioxythiophene) polystyrene sulfonate (D-Sorbitol-PEDOT: PSS) (4, FIG. 6), glycidyl methacrylate modified hyaluronic acid (5, HA-GMA; FIG. 7), and may further include 6, N-isopropyl acrylamide (NIPAM) (FIG. 9).

D-Sorbitol PEDOT: PSS 4 is also termed herein a “hydrogel,” meaning that the co-polymer is hydrophilic but water insoluble. D-Sorbitol PEDOT: PSS is electroconductive, [2][3] which is a utilized in the transmission of electric charges from the piezoelectric charges from the CFO/BTO nanobeads (7a) to the quantum dot sources in the outer layer. PEDOT: PSS coating may be formed analogously to the method provided in Kim et al., [4] by the reaction of D-sorbitol with PEDOT: PSS under reducing conditions.

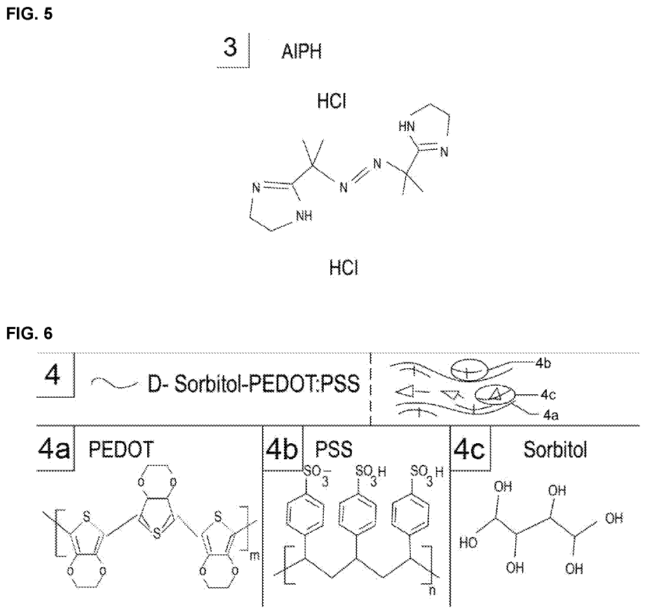

Heterogeneous intermediate layer 9 may also include glycidyl methacrylate modified hyaluronic acid 5 (HA-GMA; FIG. 7), and N-isopropyl acrylamide 6 (NIPAM) (FIG. 9). The 4, 5, 6 layer may be further impregnated with an azobis free radical generating compound such as 2,2′-Azobis(2-(2-imidazolin-2-yl) propane) dihydrochloride 3 (AIPH) (FIG. 5), a free radical precursor, and amino dextran coated-iron oxide hybrid nanoparticles (2a) (FIG. 4).

HA-GMA may comprise one or more of 5a, 5b, and 5c (FIG. 7). Hyaluronic acid (HA, 5a) is a disaccharide polymer and a component of the extracellular matrix and plays roles in inflammatory response, tissue regeneration and cell migration, among others. In this application, it may act as a biomaterial scaffold for the hydrogel, particularly utilizing crosslinking with glycidyl methacrylate (GM) 5b and NIPAM 6. GM (5b), can act as a monomer to provide epoxy functionalization to acrylate resins and promotes crosslinking in polymers and resins. It is likewise used in biocompatible hydrogels such as glycidyl methacrylate dextran. Structure 5c is an expanded view of HA-GMA/GMHA 5, showing the chemical structure of the monomer unit hyaluronic acid-glycidyl methacrylate/glycidyl methacrylate hyaluronic acid (HA-GMA/GMHA). Although HA-GMA/GMHA is a specific embodiment of a suitable biocompatible hydrogel, other polymers may be useful for this purpose.

In an embodiment, HA-GMA is part of the copolymer's structural support, as well as a component that shifts the lower critical solution temperature (LCST, also termed the “lower consolute temperature”) of the whole copolymer. The LOST is the critical temperature below which the components of a mixture are miscible for all proportions. At temperatures below LCST, the system is completely miscible in all proportions, whereas above LCST partial liquid miscibility occurs. In this case, the combination of the copolymer's components will have a target LCST above 37° C., corresponding to the hyperthermal environment of a tumor. The biocompatible polymer such as HA-GMA/GMHA can adjust the LOST either up or down so that the LOST is at least 37° C.

The NIPAM/pNIPAM (polymeric NIPAM) is meant to have similar structural and LCST-shifting roles as HA-GMA, but also undergoes a reversible LCST phase transition from a soluble hydrated state (hydrophilic) to an insoluble dehydrated state (hydrophobic) when heated above its cloud point temperature. This is the method of action for the expulsion of the hydrogel contents, triggered by a combination of the hyperthermal/slightly acidic tumor environment and NIR fluorescence.

It may be desirable to adjust the LOST of the hydrogel layer to about 37° C. Sun [5] reported (in Sec. 3.3 therein) that at HA/GMA/NIPAM polymers with AIPH incorporated into the polymer shrink when heated above the LOST, which causes ejection of embedded AIPH and generation of cytotoxic free radicals. If the LOST is too low, the hydrogel will not effectively carry incorporated active agents, such as azobis compounds, to the site of a tumor or cancer cell. Sun reports [5] that HA-GMA incorporated into polymeric NIPAM hydrogels increased the LOST, and could be adjusted to an LCST of 37° C. Thus, upon heating above 37° C. by the mechanical vibrational energy as provided herein, the polymers are expected to shrink and release active agents.

Outer layer 10 of the nanoparticle assembly may comprise one or more of immunological factors, IONP nanoparticles, and quantum dot assemblies. The iron oxide nanoparticles (IONP) 2a (FIG. 4) may have an iron oxide (Fe₃O₄) core and an amino-dextran coating, in which a hydroxy group on a dextran is functionalized with a free amino group. [6] Amino-dextrans are available in a wide range of molecular weights, ranging from about 3.5 kDa to 2000 kDa. [7] The free amino group allows for a wide range of functionality for conjugating other components.

In an embodiment, the 2a nanoparticles of this invention may include at least two different conjugated moieties (FIG. 4). One conjugated moiety may be aminooxyacetic acid (AOA) subunits (2b), linked to 2-¹⁸fluoro-2-deoxyglucose (¹⁸F-FDG) subunits (2c) (FIG. 4). AOA may be derived from [(tert-butoxycarbonyl)aminooxy]acetic acid (N-Boc-AOA) (2e). [8] This can be useful to localize ¹⁸F, which can be visualized with positron emission tomography CT, to tumor sites for visualization of tumors.

Another conjugated moiety may be a polyethylene glycol chain (PEG, 2d) (FIG. 4), for example with a MW of 2000-10000. The PEG tail may diminish the immunoreactivity of the whole assembly until activated, since PEGylated materials are known to have reduced renal clearance. [9] The combined Fe₃O₄-amino-dextran nanoparticle core 2a and AOA-¹⁸F-FDG (2b-2c) tail and PEG (2d) tail are termed herein dextran-iron oxide nanoparticles 2. The hydrophilic PEG and ¹⁸F-FDG tails may project into the outer shell 10, so that the iron oxide core 2a resides in the hydrogel layer 9 while the tail units of 2 form part of shell 10 (FIGS. 1 and 2).

AOA (2b, FIG. 4) also has potential anticancer activity, by inhibiting aspartate aminotransferase (AAT) which is highly expressed in breast adenocarcinoma, and has been postulated to be a target for antineoplastic therapies. [10]

The inventive nanoparticle assemblies 100 thus have three independent operational modalities for selectively killing cancer cells: (1) generation of cytotoxic free radicals from a vibrational effect that triggers fluorescence from quantum dots that causes photosensitizers to generate free radicals; (2) generation of cytotoxic free radicals by thermolysis of free radical precursors in a low oxygen environment; and (3) release of immunological factors or STING agonists in tissues incorporating malignant cells.

### Magnetostriction

Magnetostriction is a property of magnetic materials that such materials to change their shape or dimensions during the process of magnetization. Magnetostriction is caused from ferromagnetic materials which have an internal structure divided into domains, each of which is a region of uniform magnetization. When a magnetic field is applied, the boundaries between the domains shift and the domains rotate; both effects cause a change in the material's dimensions. The reason that a change in the magnetic domains of a material results in a change in the material's dimensions is a consequence of magnetocrystalline anisotropy: more energy is required to magnetize a crystalline material in one direction than in another. If a magnetic field is applied to the material at an angle to an easy axis of magnetization, the material will tend to rearrange its structure so that a low energy axis is aligned with the field to minimize the free energy of the system. Since different crystal directions are associated with different lengths, this effect induces a strain in the material and changes to the external dimensions of the material.

Magnetostriction is responsible for the low-pitched humming sound that can be heard coming from transformers—the hum is caused by oscillating AC currents producing a changing magnetic field that creates internal vibrations in the transformer.

Here, the 2a and the 7a nanobeads have magnetostrictive behavior and will vibrate on the application of an AC magnetic field. Both iron oxide and CoFe₂O₄ are well known magnetostrictive materials. [11]

### Quantum Dot Source

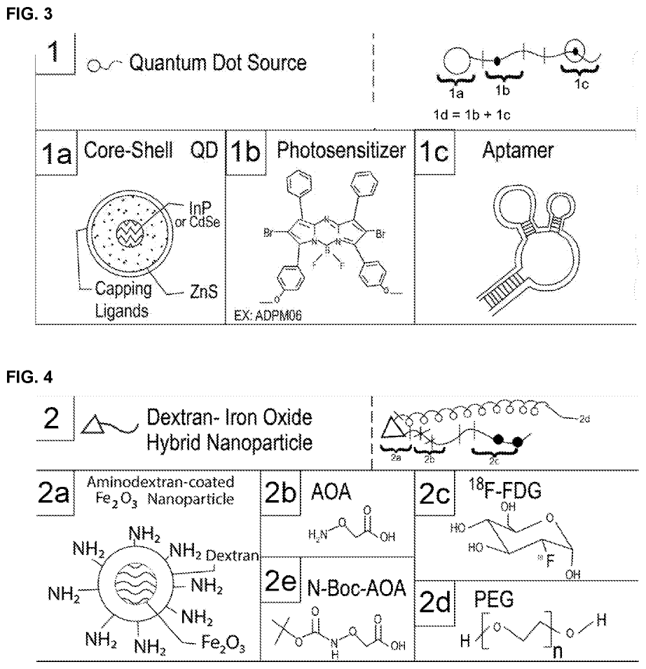

Outer layer 10 may also include a quantum dot source conjugated to a photosensitizer that may be a cancer cell targeting moiety. The quantum dots may be an electroluminescent indium phosphide/zinc sulfide (InP/ZnS or CdSe/ZnS) quantum dot source (1) with emission peaks at 683 and 785 nm, for the cellular effect (discussed below) and a peak at 763 nm peak for the vascular effect (discussed below). In an embodiment, the quantum dot source includes a quantum dot (1a, FIG. 3), with a tail (1d) conjugated thereto, wherein the tail may have one or more photosensitizer moieties (1b), and one or more aptamer moieties (1c), and associated linking groups. Aptamers of value in this invention may be prepared, for example, by the method of Dembowski and Bowser. [12]

As used herein, the term “quantum dot” (QD) refers to a semiconductor crystal with size dependent optical and electrical properties along at least three orthogonal dimensions. [13] A quantum dot is differentiated from a quantum wire and a quantum well, which are crystals with size-dependent optical and electronic properties along at most two or one dimension respectively.

Quantum dots can exist in a variety of shapes, including but not limited to spheroids, rods, disks, pyramids, cubes and a plurality of alternative geometric and non-geometric shapes. While these shapes can dramatically affect the physical, optical and electronic characteristics of the quantum dot, the specific shape does not bear on the qualification of the crystal as a quantum dot.

For convenience, the size of quantum dots can be described in terms of “diameter.” In the case of spherically shaped quantum dots, diameter is used as is commonly understood. For non-spherical quantum dots, the term diameter, unless otherwise defined, refers to the radius of revolution in which the entire non-spherical quantum dot would fit.

A quantum dot may comprise a core of one or more first materials and can optionally be surrounded by a shell of a second material. A quantum dot core surrounded by a shell is referred to as a “core-shell” quantum dot (FIG. 3, 1a).

The term “core” refers to the inner-portion of the quantum dot such that the core-region is substantially a single homogeneous monoatomic or polyatomic material. The core can be either crystalline, polycrystalline or amorphous. The core may be defect free or contain a range of defect densities. In this case, “defect” refers to any crystal stacking error, vacancy, insertion or impurity entity (e.g. dopant) placed within the core-material. Impurities can be either atomic or molecular.

While the core may herein be referred to as “crystalline”, the surface of the core may be polycrystalline or amorphous and this non-crystalline surface may extend a measurable depth within the core. The potentially non-crystalline nature of the “core-surface” does not change what is described herein as a substantially crystalline core. The core-surface region optionally contains defects or impurities. The core-surface region will preferably range in depth between one and five atomic-layers, and may be either substantially homogeneous, substantially inhomogeneous or continuously varying as a function of position within the core-surface region.

Quantum dots may optionally comprise a shell of a second material that surrounds the outside of the inner core. A “shell” is a layer of material, either organic or inorganic, that covers the surface of the core region of the quantum dot. A shell may be crystalline, polycrystalline or amorphous and optionally comprises dopants or defects. The shell material is preferably an inorganic semiconductor with a bandgap that is larger than the core material. In addition, preferred shell materials have good conduction and valence band offsets with respect to the core such that the conduction band is desirably higher and the valence band is desirably lower than those of the core. Alternatively, the shell material may have a bandgap that is smaller than that of the core material, and/or the band offsets of the valence or conduction bands may be lower or higher, respectively, than those of the core. The shell material may be optionally selected to have an atomic spacing close to that of the core material.

Shells may be “complete,” indicating that substantially all surface atoms of the core are covered with shell material. Alternatively, the shell may be “incomplete” such that only partial coverage of the core atoms is achieved. In addition, it is possible to create shells of a variety of thicknesses, defined in terms of the number of “monolayers” of shell material that are bound to each core. “Monolayer” is a term known in the art referring to a single complete coating of a shell material (with no additional material added beyond complete coverage). In the current invention, shells will preferably be of a thickness between 0 and 10 monolayers, where it is understood that non-integer numbers of monolayers correspond to the state in which incomplete monolayers exist. Incomplete monolayers may be either homogeneous or inhomogeneous, forming islands or clumps of shell material on the surface of the quantum dot. Shells may be either uniform or nonuniform in thickness. In the case of a nonuniform thickness shell, it is possible to have an “incomplete shell” that contains more than “one monolayer” of shell material. Finally, shell thickness will preferably range from about 1 Å to 100 Å.

Quantum dots may have an interface region between the core and shell. The interface may comprise an atomically discrete transition between the material of the core and the material of the shell or may comprise an alloy of materials between the core and shell. The interface may be lattice-matched or unmatched and may be crystalline or amorphous. The interface may contain defects or be defect-free and may contain impurities. The interface may be homogeneous or nonhomogeneous and may comprise chemical characteristics that are graded between the core and shell materials such that a continuous transition is made between the core and shell. Alternatively, the transition can be discontinuous. The width of the interface region can range from an atomically discrete transition to a continuous graded alloy of core and shell materials that are only purely core material in the center of the quantum dot and purely shell material at the outer surface. In an embodiment, the interface region will be between one and five atomic layers thick.

A shell may optionally comprise multiple layers of a plurality of materials in an onion-like structure, such that each material acts as a shell for the next-most inner layer. Between each layer there is optionally an interface region. The term “shell” is used herein to describe shells formed from substantially one material as well as multi-layer shells.

A quantum dot may optionally comprise a “ligand layer,” comprising a plurality of organic molecules bound either covalently or non-covalently to the outer surface of the quantum dot. A quantum dot comprising a ligand layer may or may not also comprise a shell. As such, the organic ligands of the ligand layer may bind to either the core or the shell material or both (in the case of an incomplete shell). The ligand layer may comprise a single molecular species, or a mixture of two or more molecular species. Each molecular species will have an affinity for, and bind selectively to, the quantum dot core, shell or both at least at one point on the molecule. The molecular species may optionally bind at multiple points along the molecule. The molecular species may optionally contain additional active groups that do not interact specifically with the surface of the quantum dot. The molecular species may be substantially hydrophilic, substantially hydrophobic or substantially amphiphilic. In general, the molecular species can be an isolated organic molecule, a polymer (or a monomer for a polymerization reaction), an inorganic complex, and an extended crystalline structure.

When referring to a population of quantum dots as being of a particular “size”, what is meant is that the population is made up of a distribution of sizes around the stated “size”. Unless otherwise stated, the “size” used to describe a particular population of quantum dots will be the mode of the size distribution (i.e. the peak size). For purposes of this invention, the “size” of a quantum dot will refer to the diameter of the core material. If appropriate, a separate value will be used to describe the “shell-thickness” surrounding the core. For instance, a 3 nm indium-phosphide (InP) quantum dot with a 1.5 nm zinc sulfide (ZnS) shell is a quantum dot would be a 3 nm diameter core surrounded by a 1.5 nm thick shell, for a total diameter of 6 nm.

A feature of quantum dots is that they usually have a small size with a large surface area per unit volume. QD's further exhibit quantum confinement effects, and thus have different physicochemical characteristics from the characteristics of the bulk material. Quantum dots may absorb light from an excitation source and may emit light energy corresponding to an energy bandgap of the quantum dot. In the quantum dots, the energy bandgap may be selected by controlling the sizes and/or the compositions of the nanocrystals. Thus, for a given crystal composition, changing the size of the crystal during preparation will result in different emission wavelengths. QDs generally have desirable photoluminescence properties and have a high color purity. Therefore, QD technology is used for various applications, including display devices and bio-light emitting elements.

When the quantum dots are activated for example by illumination with by UV light, an electron in the quantum dot can be excited to a state of higher energy. In the case of a semiconducting quantum dot, this process corresponds to the transition of an electron from the valence band to the conductance band. The excited electron can drop back into the valence band releasing its energy by the emission of light as fluorescence. The color of that light depends on the energy difference between the conductance band and the valence band. The wavelength of the light can be tuned by changing the size of the crystal during manufacture of the quantum dots.

In an embodiment, the electron excitation and emission in a quantum dot can be activated by an electric current. In this embodiment, an electric current can boost an electron from a valence band to a conductance band, causing light emission when the electron drops back to its valence band. The technique has been used in the development of light emitting diodes and video displays. See for example U.S. Pat. No. 9,933,351. The electric current needed to activate the quantum dots may come from the piezoelectric effect on CFO-BTO nanobeads 7a vibrating under the influence of an AC magnetic field [14]. The current from the piezoelectric effect may be transmitted through the electroconductive heterogenous hydrogel layer 9.

In an embodiment, quantum dot source 1 is a multicomponent subassembly, comprising a quantum dot having a core-shell sphere 1a with a core of InP or cadmium selenide (CdSe), a shell of ZnS, and an external capping ligand layer providing further chemical stability and functionalization. In an embodiment, InP may be preferred as CdSe may be of concern in therapeutics because cadmium is toxic. [15] Quantum dot source 1 may further include chains conjugated to quantum dot 1a including one or more photosensitizer linkages 1b and one or more aptamers 1c. [16] InP/ZnS quantum dots are environmentally-friendly, heavy metal-free, and non-toxic.

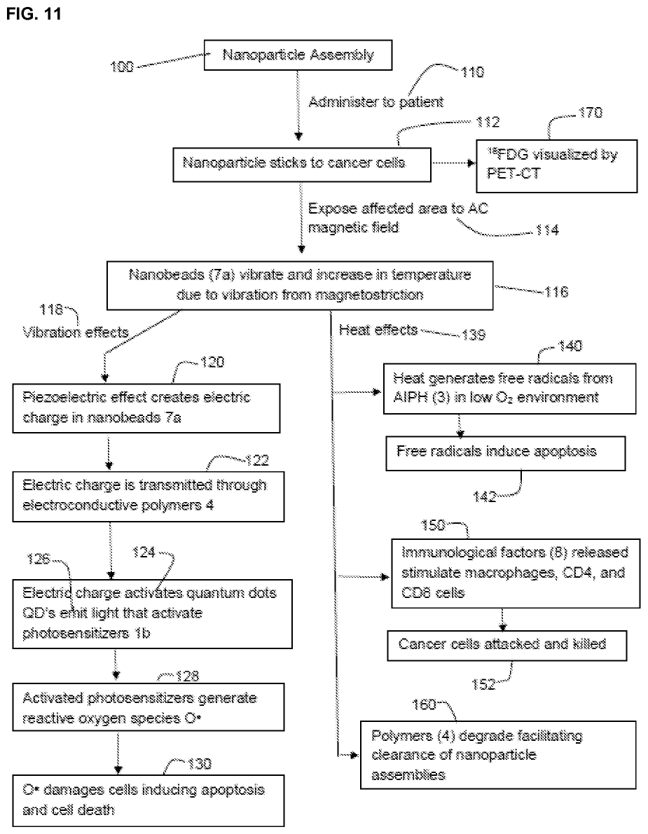

An InP/ZnS QD may alternatively be a component in a quantum-dot light-emitting diode (QLED), which have improved energy efficiency. [17] Electroluminescent QLED devices can emit specific wavelengths based on carrier injection, which could either be electrons or holes depending on the quantum dots and the injection layer (the analogue of which would be the conduction layer of the nanoassembly 100). [17] This feature also validates 124 in patent FIG. 11.

### Photosensitizers

Photosensitizers produce a physicochemical change in a neighboring molecule by either donating an electron to the substrate or by abstracting a hydrogen atom from the substrate. At the end of this process, photosensitizers usually return to a ground state, where the molecule remains chemically intact. In this invention, photosensitizers produce a cytotoxic effect when irradiated with electromagnetic energy (light) of an appropriate wavelength by generating singlet oxygen (also termed a “reactive oxygen species” or ROS). Singlet oxygen is highly reactive and is toxic to a proximal cell or target organism. ROS's such as for example, hydroxyl (HO·), peroxide (O₂²⁻), singlet oxygen (¹O₂), and superoxide (·O²⁻), play a crucial role in homeostasis and cell signaling in biological processes. A high level of ROS's can lead to cell death by inducing oxidative damage to proteins, lipids, DNA, and dysfunctions in cell metabolism. [18]

A molecular conjugate containing a photosensitizer should efficiently absorb electromagnetic energy of the appropriate wavelength with high quantum yield to efficiently generate the energized form of the photosensitizer. Toxicity to the target organism should increase substantially, preferably 10-fold, 100-fold, or even 1,000-fold upon irradiation. Ideally, a photosensitizer will exhibit low background toxicity, i.e., it should not be toxic in the absence of radiation with energy of the appropriate wavelength. Further, a useful photosensitizer should be readily soluble in a variety of solvents, including those in which it is coupled to the targeting moiety and those in which it is administered to a subject. The context of solubility will differ depending on the conditions in which the photosensitizer is coupled to form a conjugate (or the conditions in which the conjugate is administered). For example, the photosensitizer and targeting moiety may be coupled in a reaction requiring solubility in DMSO, water, ethanol, or a mixture thereof (e.g., a 1:1 mixture of DMSO:H₂O or 5% ethanol in water).

Photosensitizers include, for example, hematoporphyrins, such as hematoporphyrin HCl and hematoporphyrin esters [19]; dihematophorphyrin ester [20]; hematoporphyrin IX and its derivatives. [21]. A family of Boron dipyrromethene (BODIPY) has been disclosed. [22] ADPM06 is a member of the BODIPY class of photosensitizers.

Awuah and You [22] confirm the excitation wavelength ranges for various photosensitizers overlapping with QD emission wavelengths discussed above. Awuah and You also confirm activation of photosensitizers based on fluorescence, for photosensitizers including the BODIPY class.

The QD's, when activated and fluorescing, are designed to activate one or more photosensitizer materials in the inventive nanodevice. QD's are known to able to activate photosensitizers [23]. The photosensitizers in turn generate a cytotoxic reactive oxygen species that causes cellular damage and induces apoptosis, killing tumor cells. In an embodiment, the photosensitizers in this invention may be conjugated to a quantum dot, as a kind of tail piece (1d).

In an embodiment, the photosensitizer 1b may be a BODIPY structure such as azadipyrromethene/ADPM06, or a palladium-substituted bacteriochlorophyll derivative such as padeliporfin, porfimer, or tin ethyl etiopurpurin. Azadipyrromethene/ADPM06 is a nonporphyrin photodynamic therapeutic (PDT) agent having a cellular effect. Other photosensitizers are within the scope of this invention. Representative examples are shown in Table 1.

TABLE 1
Representative Photosensitizers

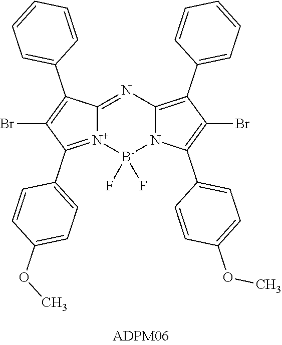 [SMILES: COc1ccc(C2=[N+]3C(=Nc4c(-c5ccccc5)c(Br)c(-c5ccc(OC)cc5)n4[B-]3(F)F)C(c3ccccc3)=C2Br)cc1]

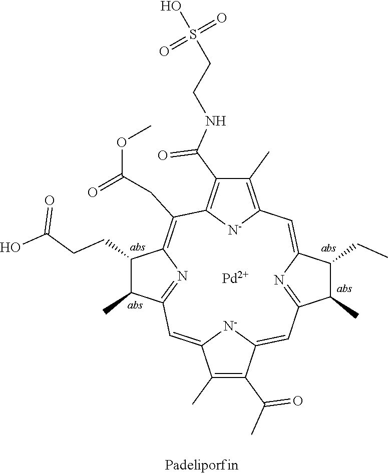

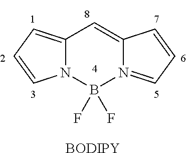

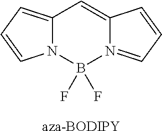

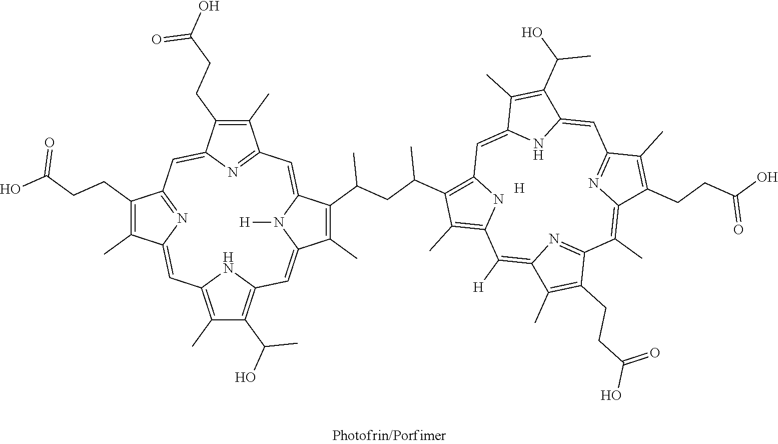

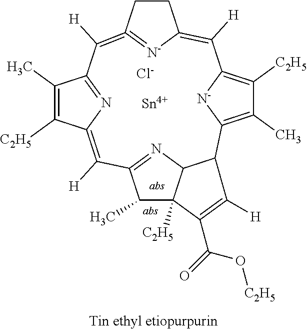

When activated, ADPM06 exhibits IC₅₀ values in the micromolar range in human tumor cells and induces apoptosis, killing the cells. This is termed herein a “cellular effect.” The palladium-substituted bacteriochlorophyll derivative/padeliporfin photosensitizer is specific for epithelial cells. The action of padeliporfin is termed here a “vascular effect.” The differentiation between vascular and cellular effect is a matter of broad target cell type: “cellular effect” refers to nanoparticles designed to target and attack distinct tumor cells, which has a broader range of target types depending on the site of the tumor (liver, pancreas, brain, etc). “Vascular effect,” on the other hand, refers to nanoparticles designed to specifically target endothelial cells of a tumor vasculature.

Padeliporfin is approved in Europe and sold under the brand name Tookad, for treating men with low-risk prostate cancer. ADPM06 is a dye being investigated as a photodynamic agent [24, 25].

### Free Radical Precursors

The heterogenous intermediate layer 9 may be impregnated with a free radical precursor such as an azobis compound, that can generate cytotoxic free radicals thermolytically. In an embodiment, an azo compound in this invention can react to heat generated from the vibration of IONP's on exposure to an AC magnetic field and produce cytotoxic free radicals in a low oxygen environment. [26, 27] Exemplary free radical initiators for this purpose include 2,2′-Azobis(2-(2-imidazolin-2-yl) propane) 3 (AIPH) (FIG. 5), [18, 28] [29] or the dihydrochloride salt thereof. Other azo free radical precursors that may be useful in this invention and that exhibit hyperthermic degradation to free radicals include dimethyl 2,2′-azobis (2-methylpropionate) (AIBME) [27] and 2,2′-Azobis(2-amidinopropane) dihydrochloride (AAPH). [30] Structures for these compounds are shown in Table 2.

TABLE 2
Azobis Free Radical Precursors

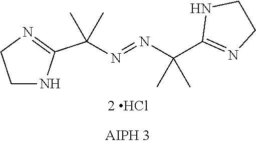 [SMILES: CC(C)(/N=N/C(C)(C)C1=NCCN1)C1=NCCN1]

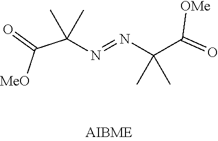 [SMILES: COC(=O)C(C)(C)/N=N/C(C)(C)C(=O)OC]

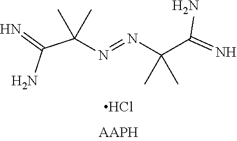

It has been suggested that cancer cells are extremely resistant to chemotherapy and oxygen-dependent photodynamic therapy in the hypoxic areas of the tumor. [18] Thus, the development of effective strategies to overcome the tumor hypoxia microenvironment and enhance the accumulation of biocompatible nanoparticles at the tumor site could provide a synergistic anticancer treatment effect. Hence, a compound deliverable into a tumor that can disintegrate and produce oxygen-independent highly toxic free radicals upon external stimulation may have good therapeutic efficacy. [27] Thus, free radicals induce cellular damage and apoptosis. By delivering the inventive components to the proximity of tissues containing malignant cells and inducing the formation of free-radicals by thermolysis, an anticancer therapeutic effect is expected.

Any of several azo free radical generating compounds that can be activated by heat are expected to be efficacious. For example, Jun Yang et al. studied (AIPH), [18] which has excellent water solubility and disintegrates promptly under the stimulation of illumination or heat to produce alkyl free radicals. See also Sun et al. [5]. Similarly, Gao et al. studied AIBME. [27] The generated free radicals are toxic to cells, immediately oxidizing cellular elements or interacting with oxygen to produce secondary toxic substances.

### Immunological Factors

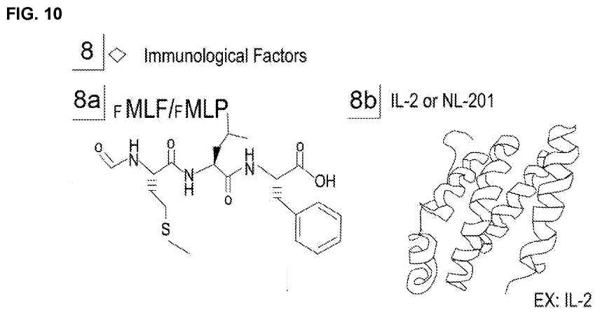

Several immunological factors are contemplated in this invention (FIG. 10). In an embodiment, immunological factors (8) such as N-formylmethionine-leucyl-phenylalanine (fMLF) (8a) and neoleukin-2/15 (Neo-2/15, NL-201, or some other IL-2-like derivative) (8b) [31-33] may be incorporated into the hydrogel copolymer. In an embodiment, during the activation of the assembly and resulting phase change of the copolymer layer (4), 8a and 8b will be released. 8a and 8b are intended to stimulate macrophages (MP's), CD4, and CD8 cells for the ends of secondary stimulation of the immune system and cellular debris clearance via simulated cytokine pathway/simulated necrosis signal. See also FIGS. 11 150 and 152. The 8a factors act primarily as macrophage activators, whereas the 8b factors act as stimulators of T-cell differentiation and enhancers of natural killer cells and cytotoxic T-cells.

Another immunological factor embodiment is an agonist of the cyclic GMP-AMP synthase (cGAS) and stimulator of interferon genes (STING) pathway. [34] STING is a transmembrane protein localized to the endoplasmic reticulum which functions as an adaptor protein in the cGAS-STING pathway. cGAS-STING is a cytosolic DNA-sensing pathway that drives activation of type I interferon (IFN) and other inflammatory cytokines in the host immune response against tumors. Recognition of cytoplasmic tumor-derived DNA by c-GAS generates cGAMPs which are natural ligands of STING protein. The binding of cGAMP to STING induces transformational changes in STING protein, activating a downstream signaling cascade involving TBK1 and IRF-3, which results in the production of type I IFNs. [34] The therapeutic effects of many anticancer modalities, including immunotherapies, depend to a large degree on type I IFN signaling. Type I IFNs exert their anti-tumor effects by inhibiting tumor proliferation and enhancing the expression of MHC class I required for recognition by CD8+ T cells. A number of STING agonists are known with anticancer activity. [34-36]

Another enzyme, ectonucleotide pyrophosphatase/phosphodiesterase 1 (ENPP1), is believed to play an important role in the immunological responses to various stimuli through the cGAS-STING pathway. [37] ENPP1 is a transmembrane phosphodiesterase known for its central role in purinergic signaling. ENPP1 can downregulate cGAS-STING signaling by hydrolyzing cGAMP, the natural STING ligand. Damage associated molecular patterns (DAMPs) as well as pathogen associated molecular patterns (PAMPs) activate the immune system via STING. cGAS senses cytosolic DNA and catalyzes the conversion of GTP and ATP to cyclic GMP-AMP (cGAMP). Subsequently, 2030-cGAMP activates STING to initiate an inflammatory response via the TANK-binding kinase 1 (TBK1)-Interferon Regulatory Factor (IRF) 3 pathway to produce type 1 interferons (IFNs) and other cytokines. A link between the cGAS-STING pathway and ENPP1 has emerged whereby the hydrolysis of cGAMP by ENPP1 attenuates cGAS-STING signaling. [37] Thus, ENPP1 antagonists are expected to upregulate STING and may exert an antitumor effect. A number of ENPP1 antagonists have been disclosed in recent papers and patents. [34, 37-39]. Representative examples of STING agonists and ENPP1 inhibitors are listed in Table 3.

TABLE 3
STING Agonists/ENPP1 Inhibitors

ADU-S100/MIW815-STING Agonist
SR-8541A-ENPP1 Inhibitor
SR-8314-ENPP1 Inhibitor
MK-1454-STING Agonist
SB11285-STING Agonist
BI-STING (BI 1387446)-STING Agonist

Many types of cancers can induce a spontaneous adaptive T cell response and foster an immunosuppressive microenvironment favoring its development. Therefore, targeting the cGAS-STING-TBK1 (TBK1, TANK-binding kinase 1) pathway by using agonists to “heat up” tumor microenvironment via secretion of IFNs and other cytokines could enhance anti-tumor immune response. [40] Ding discloses natural and synthetic cyclic dinucleotides (CDNs) and other compounds as STING agonists. [40] Regulation of the cGAS-STING pathway is discussed by Gao et al. [41]

STING agonists and ENPP1 inhibitors exert their effects locally, within cells. Thus, delivering agents directly to the site of tumors or other cancer cells may be an advantageous modality for killing cancer cells without exposing normal cells to cytotoxic agents. In view of the above discussion, embodiments of this invention may include STING agonists or ENPP1 antagonists embedded in the heterogenous intermediate layer 9, that are releasable in the proximity of a tumor or cancer cell in accordance with this invention. Thus, the thermolysis of nanobeads 7a causes the heterogenous layer 9 to change polarity which expels immunological factors in the local environment of malignant cells.

### Aptamers

Aptamers are single-stranded DNA (ssDNA) or RNA (ssRNA) oligonucleotide ligands that specifically bind to various molecular targets, and the use of aptamers as biomaterials, diagnostic and therapeutic tools, or for the development of new drug delivery systems has been investigated in numerous studies. [42] Aptamers can discriminate between closely related targets with high specificity and affinity (dissociation constant [K_{D}]=pM˜nM). In addition to the specific recognition of their targets, aptamers also possess several advantages over antibodies, such as high stability, ease of synthesis, low immunogenicity, and diverse target. Moreover, aptamers are amenable to chemical modification and bioconjugation to various moieties, such as nanoparticles, imaging agents, small interfering RNAs (siRNAs), and therapeutic drugs. Based on all of these advantages, aptamers are considered to be an alternative to antibodies and to have great potential as molecular probes for cancer diagnosis and treatment.

The synthesis of aptamers is based on an in vitro evolution procedure, known as “Systematic Evolution of Ligands by Exponential Enrichment” (SELEX). [43] The strategy involves the iterative selection of high-affinity nucleic acid ligands towards a broad range of targets, including small molecules, proteins, peptides, toxins, whole cells, and tissues. Aptamers have been reported for cancer diagnosis, infectious disease diagnosis, and as therapeutic agents. [43], [12]. Kim et al. used this SELEX method to identify aptamers that bind to tumor-initiating cells (TIC) in glioblastomas. [44]

In an embodiment, the quantum dot source 1 has a tail moiety including an aptamer 1c (FIG. 3). A surface aptamer (1c) may terminate the photosensitizer chains with highly selective binding targets. In an embodiment, the aptamer binds to a receptor overexpressed on cancer cells. For example, an aptamer may target annexin A1, which is upregulated in certain cancers, [45] in particular endothelial cancers for the vascular effect.

In another embodiment, the nanoparticle assembly 100 includes aptamers linked to the dextran iron oxide nanoparticles 2 as part of outer layer 10.

### Iron-Oxide Nanoparticle Subassemblies/Theranostic Features

Outer layer 10 may also include vascular-effect aminooxyacetic group-conjugated dextran-iron oxide hybrid nanoparticle subassemblies (2) (also termed herein “IONP's”), [27] with a core of an amino dextran coated-iron oxide hybrid nanoparticle (FIG. 4, 2a) that may be PEGylated by conjugated to a first tail moiety including polyethylene glycol (2d) (MW 2000-10000). PEGylation changes the physical and chemical properties of a drug molecule, such as its conformation, electrostatic binding, and hydrophobicity, and can result in an improvement in the pharmacokinetic behavior of the drug. In general, PEGylation improves drug solubility and decreases immunogenicity. PEGylation also increases drug stability and the retention time of the conjugates in blood, and reduces proteolysis and renal excretion. [46]

Additional tail moieties may be present including one or more aminooxyacetic linkers (2b) and may include one or more 2-¹⁸Fluoro-2-deoxyglucose (¹⁸F-FDG) subunits (2c). ¹⁸F-FDG is taken up by cells, phosphorylated by hexokinase (whose mitochondrial form is greatly elevated in rapidly growing malignant tumors), and retained by tissues with high metabolic activity, such as most types of malignant tumors. As a result, positron emission tomography (FDG-PET) can be used to visualize tumors for diagnosis, staging, and monitoring treatment. [47] PET scanners are frequently combined with computed tomographic scanners (PET-CT) scanners for a more complete visualization of internal physiologic structures. The ¹⁸F-FDG moieties provide the diagnostic features of this invention.

Additionally, a tail component of IONP 2 may include an aptamer conjugated thereto, in particular in embodiments lacking a quantum dot source.

Core 2a without the tail may also be a component of layer 9.

### Formulations

The inventive nanoassemblies 100 may be provided as a colloid or suspension in water and one or more additional excipients for use as a therapeutic. In an embodiment, the colloid or suspension is provided in an injectable formulation.

### Nanoparticle Assembly

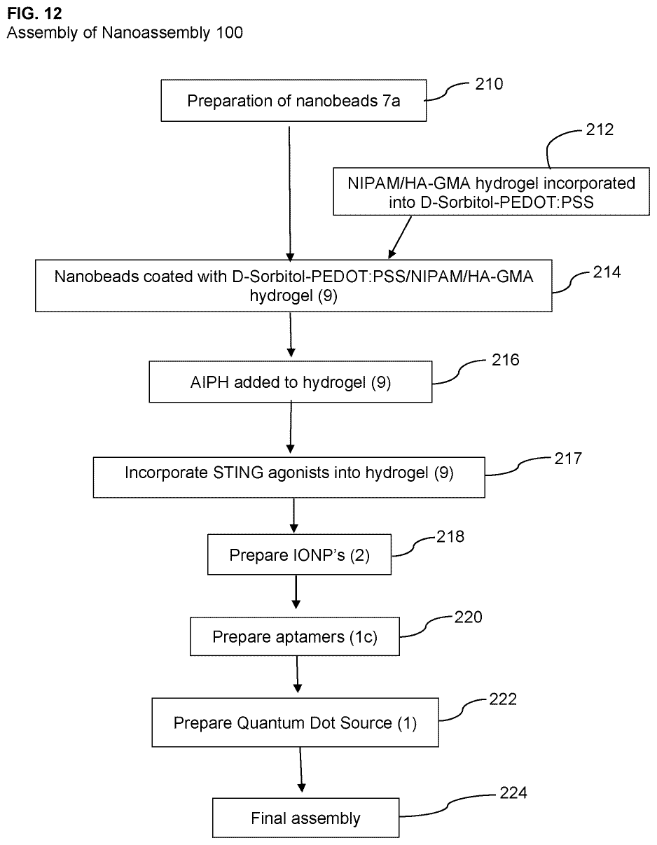

The inventive nanoparticle may be prepared by methods as discussed herein. A flowchart with a representative assembly method is shown in FIG. 12.

In an embodiment, the CFO/BTO nanobeads (7a) may be prepared by the method of Rao. [14] (FIG. 12, 210) CFO can be prepared by cobalt (II) chloride hexahydrate (CoCl₂·6H₂O), ferric chloride hexahydrate (FeCl₃·6H₂O), in the presence of NaOH which precipitated CoFe₂O₄ particles about 25 nm in diameter. BTO was formed by Ba-acetate and titanium isopropoxide. CFO/BTO nanobeads are formed by suspending CFO in water and mixing in a sol (colloid) of BTO to form a gel. The gel is dried and calcined at 700° C. for 2 h, and then recalcined at 800° C. for 2 h to obtain the nanocomposite. During calcination, the BTO nanoparticles will grow around the dispersed CFO nanoparticles to form nanoparticles with a CFO core coated by BTO. [14]

The 7a nanobeads may be coated with PEDOT: PSS to form hydrogel layer (9) by modifications of the method of Kim et al. [4] (FIG. 12, 214). Kim disclosed selenium nanoparticles coated with PEDOT: PSS, but this method may be adapted to the CFO/BTO nanobeads. Thus, a dilute solution of D-Sorbitol-PEDOT: PSS can be reacted with CFO/BTO nanoparticles and this is expected to form a uniform coating of D-Sorbitol-PEDOT: PSS as heterogenous layer (9). D-Sorbitol-PEDOT: PSS can be prepared from PEDOT: PSS as described by Onorato et al. [3] Onorato et al. showed that sorbitol oxidizes to 1,6-anhydrosorbitol in PEDOT: PSS on heating, and the D-sorbitol doped PEDOT: PSS, whether or not the D-sorbitol is oxidized to 1,6-anhydrosorbitol, has substantial improved conductivity as compared to PEDOT: PSS without added D-sorbitol.

Heterogenous layer (hydrogel) 9 components AIPH (3), HA-GMA (5), and NIPAM (6) may be incorporated into layer 9 by the methods of Sun. [5] Sun discloses a localized injectable hydrogel combining the photothermal therapy (PTT) and the thermodynamic therapy (TDT) incorporating AIPH (3), HA-GMA (5), and NIPAM (6). Sun discloses that a cross-linking reaction between the HA-GMA and NIPAM can be initiated in a redox system through the polymerization of double bonds on each monomer to form an HA-GMA/NIPAM hydrogel. The HA-GMA/NIPAM hydrogel can be blended with D-Sorbitol-PEDOT: PSS (FIG. 12, 212).

After the nanobead coating step (214), AIPH (3) can be additionally added to this PEDOT: PSS hydrogel as described by Sun [29] (FIG. 12, 216).

Immunological factors 8 may be incorporated into the hydrogel of heterogenous layer 9 by the methods of Wilson et al. [48] or Shae et al. [49] (FIG. 12, 217). Wilson reported that intratumoral stimulation of STING may potentially synergize with immune checkpoint inhibitors and that cyclic dinucleotides (CDNs) such 2,5 linked cGAMP have been shown to activate interferon regulatory factor 3 (IRF3) and directly bind STING and subsequently initiate TBK1-IRF3 and NFkB dependent type I IFN proinflammatory immune responses which have antitumor activity. Wilson et al. prepared nanoparticles based on poly (beta-amino esters) (PBAEs), a class of synthetic, cationic polymers, have been found to be effective as non-viral gene delivery agents for a wide variety of cell types both in vitro and in vivo for cytosolic delivery of anionic CDN molecules such as cGAMP. [48] Shae et al. likewise describes a STING-activating nanoparticle (STING-NP) based on polymer vesicles (polymersomes) engineered for efficient cytosolic delivery of cGAMP, an agonist of STING. Through control of polymer properties, formulation methodologies and an in situ vesicle membrane crosslinking strategy, cGAMP is efficiently encapsulated into polymersomes that disassemble in response to endolysosomal acidification to unveil membrane-destabilizing polymer segments that promote endosomal escape of cGAMP. [49] Thus, incorporation of cGAMP or other STING agonists [34-36] may be deployed in the instant invention using the methods disclosed in Wilson et al. [48] or Shae et al. [49].

Alternatively, ENPP1 immunological factors can be incorporated into the hydrogel by the methods of Wilson et al. [48] or Shae et al. [49], since many ENPP1 inhibitors are nucleotides and structurally similar to CDN STING agonists. [37] Other ENPP1 inhibitors are not nucleotides but may be incorporated into hydrogel layer 9 by similar methods as discussed here for STING agonists or azobis compounds. Liu et al. [50] discloses general methods at Sec. 3.2, Table 3, and FIG. 4 for incorporating drugs suce has non-nucleotide ENPP1 inhibitors into hydrogels that may be useful in this invention.

Iron oxide nanoparticles (IONP's) can be linked to ¹⁸F-FDG using the method of De Simone [51] according to Equation 1 linking ¹⁸F-FDG to functionalized IONP's via an oxime (FIG. 12, 218).

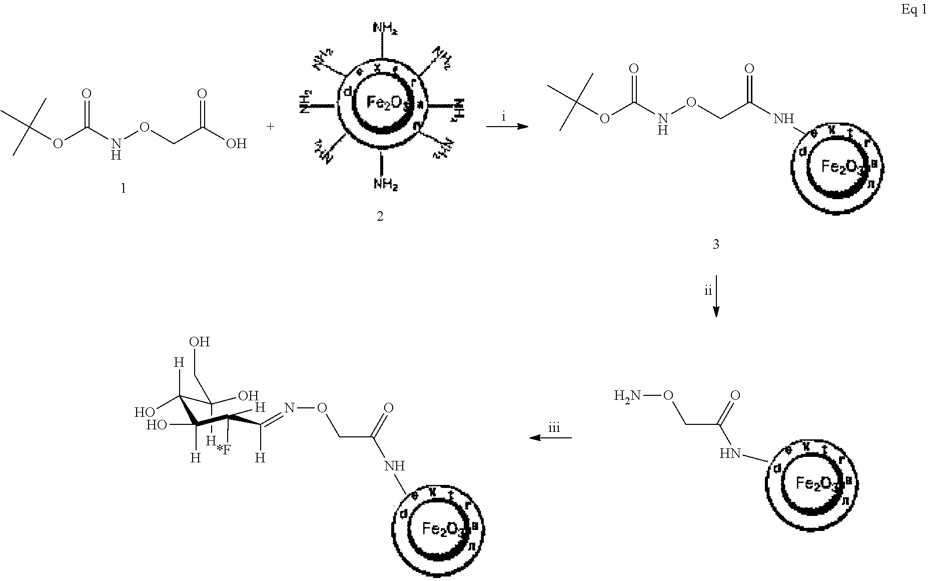

Aptamers 1c in this invention can be obtained (FIG. 12, 220) by the method of Dembowski and Bowser [12] which discloses incubating a randomized nucleic acid pool with a target. Sequences with affinity for the target are separated, amplified, and incubated again in an iterative process. The step of separating binding from non-binding sequences uses capillary electrophoresis (CE) which has good resolving power and stringent yet flexible selection conditions allowing high affinity aptamers to be obtained in only 2-4 rounds of selection.

Quantum dot source 1 with tail 1d which may include a photosensitizer 1b and aptamer 1c can be prepared by the methods disclosed in Labiadh and Hidouri [52] (FIG. 12, 222).

The assembly of colloidal or suspension nanoassemblies with magneto-fluorescent character may be accomplished by adapting the method of Chen et al., taking a ferromagnetic nanobead core region, coating it, adding quantum dots, and further coating the assembly with a polymer [53] (FIG. 12, 224). This method provides “supernanoparticles” having a superstructure consisting of a close-packed magnetic nanoparticle “core,” which is surrounded by a “shell” of fluorescent quantum dots. Chen et al. employed a silica coating rather than PEDOT: PSS, and a polyvinyl pyrrolidinone (PVP) outer layer. This method is adaptable to the inventive materials for nanoassembly 100, which has CFO-BTO nanobead core particles wrapped in a PEDOT: PSS coating and covered with a heterogenous layer of quantum dot sources 1 and dextran iron oxide hybrid nanoparticles (IONP's). The IONP's may further include a PEG chain as discussed above. Chen et al. also discloses the tunability of QD's, which may important for activation of photosensitizers.

Nanoassemblies of this invention can be constructed with only certain features and not others incorporated. For example, a nanoassembly can be constructed only containing the quantum dot source and photosensitizer, and without the heat activated azobis compounds or immunological factors. Likewise, a nanoassembly can be constructed without the quantum dot source 1 but including the heat activated azobis compounds. In another embodiment, a nanoassembly can be constructed with the quantum dot source and the immunological factors. Other combinations of features in nanoassemblies are possible that include certain features and omit other features.

### Nanoparticle Operation

This section refers generally to FIG. 11.

Upon administration of the particulate treatment (FIG. 11, 100) to a patient suffering from cancer, an uptake time period may be allowed to elapse that allows for the inventive nanoparticles to find target malignant cells or tissue and be cleared from any healthy tissue. The aptamers may assist in targeting the inventive nanoparticles to corresponding receptors that are highly overexpressed on most cancer cell types while underexpressed on many healthy cell types, allowing for selective targeting and entry into these cells (given the already “leaky” nature of tumor vasculature). Thus, the inventive particles may attach to the surface of cancerous cells. Alternatively, the inventive nanoparticles may penetrate the cell membrane and exert its effect internally to the cell.

In another alternative, DC magnetic fields can be used to guide the nanoassemblies 100 to a desired location. A DC magnetic field will not activate the magnetostrictive features of this invention. The location can be visualized using PET-CT to ensure that the nanoassemblies are adjacent to or incorporated into a tumor site.

In either case, the inventive nanoassemblies may be incorporated into malignant cells and exert their effects intracellularly, or can exert the effects as described herein extracellularly at a tumor site or in the vicinity of malignant cells. Note that intratumoral injection of therapeutic interventions is known. [34]

Once the aforementioned time elapses, administration of a sufficient strength alternating current (AC) magnetic field (114) is applied to the tumor region, inducing a magnetostrictive effect (116) within the CFO-BTO nanobeads 7a in core 7 and a heating effect (139) and vibration (118) in the nanoparticles 2a. Magnetostrictive (MS) materials change their shape or dimensions during the process of magnetization, which causes the vibration and heating of the nanobeads 7a and to a lesser extent in the IONP's 2a. [14]

IONP's 2a function as a heat transfer agent from the core 7 to the outer layers of nanoparticle assemble, and also as a carrier of FDG and in some embodiments, as a carrier of an aptamer with affinity for a surface protein of a malignant cell.

The heat and vibration of the nanobeads 7a cause up to three distinct effects in this invention: (1) activation of ROS's via piezoelectric effect that activates light emission from quantum dots that activates photosensitizers that generate ROS's; (2) heat from the mechanical vibration effect generates oxygen independent cytotoxic free radicals from free radical precursors such as azobis compounds; and (3) the heat effects may release immunological factors, STING agonists, or ENPP1 antagonists (or a combination thereof) that exert a local immune response inducing apoptosis in malignant cells. Embodiments of this invention include any combination of these effects including all three in the same nanoparticle assembly.

In a first embodiment, the vibration of nanobeads 7a should create a piezoelectric effect from nanobeads 7a which pumps charge carriers (120) through the polymer membrane (4) [3] into InP quantum dots (122). [14] The charge carriers can either be electrons or electron holes, since the electroconductive polymers transfer electrons only, rather than a type of ion. The type of charge carrier may not be specific, as the charge carrier ultimately depends on the quantum dots which can be either p-doped or n-doped to achieve the same effect.

This electric charge activates the quantum dots (124), [17] which emit light (126) at their given range adjacent to the conjugated photosensitizers. The photosensitizers then react with the emitted light and molecular oxygen to create reactive oxygen species (ROS) (128) that damage the tumor's vasculature, irreversibly damaging tumor cells over a short period of time and causing apoptosis (cell death) (130). The generation of ROS's is disclosed for example by Awuah and You [22] (see FIGS. 16 and 17 therein).

In a second embodiment arising from the magnetostrictive effect causing vibration of the IONP's 2a is heat (139) generated by vibrational energy, which causes the PEDOT: PSS hydrogel to contract. (Sun, ref. [5] see Sec. 3.5.3 therein) The compound release is caused by a combination of the phase transition of the NIPAM/HA-GMA component of the copolymer, and eventual breakdown of the copolymer, The phase transition causes a deformation of the copolymer and expels the free radical precursor and immunological inner contents through repulsive forces induced between the hydrophilic payload and now-partially-hydrophobic copolymer. An example of this is also in the Sun paper, with respect to the AIPH; expected temperatures at the target areas are above 39° C., which was also the target temperature range suggested by Sun. Magnetostriction causes some degree of frictional heating, which occurs within the CFO component of the nanobeads 7a and within the IONPs 2a.

This effect from the AC magnetic field causes magnetostriction in the nanobeads (7a), and wherein magnetic induction generates heat causing the polymer material (4) to deform and warm up, thereby generating free radicals from an azobis free radical precursor (3), and wherein the free radicals induce cellular damage and apoptosis in cancer cells.

Responding to the heat may also potentially trigger further generation of free radicals from the hydrogel; further heating can induce a breakdown of the biodegradable hydrogel, allowing for easier breakdown and removal in the body.

During the AC magnetic field activation of the assembly, the magnetostrictive effect also creates an elevated temperature in the iron-oxide nanoparticles (2a) due to vibrational energy, creating a volumetric change and phase change within the copolymer layer (4), which shifts the NIPAM (6) within the copolymer layer from hydrophilic to hydrophobic along with the volumetric change. This leads to a destabilization of the copolymer (aiding in breakdown) and an expulsion of hydrophilic materials in the layer, such as AIPH (3) and immunological factors (8).

The specific photosensitizers have enhanced performance under hypoxic conditions within the tumor given incident 683 nm light combined with a heating effect from the 2a nanoparticles within the hydrogel/copolymer, allowing for the thermal generation of free radicals from the AIPH (3) while in the absence of oxygen (140). [5] The free radicals at this stage induce apoptosis (142). Id. This mechanism is therefore particularly useful in low oxygen environments, which can occur in the interior of some solid tumors.

For both the vascular and cellular components of the treatment, the aminooxyacetic group-conjugated dextran-iron oxide hybrid nanoparticles allow for the chemical conjugation of ¹⁸F-FDG (2c, 170) to the dextran-iron oxide nanoparticle (ergo to the copolymer surface as a conjugation of the whole nanoparticle) when the treatment is desired, providing a radiolabel. Using this integrated fluorine-18, the treatment can both selectively affect and provide progressive contrast for the tumor and its constituent cells, allowing for the utilization of real-time PET-CT imaging/observation along with the administration of the treatment itself.

Upon administration of multiple of these particulates (as well as the incident magnetic field and relative hyperthermic tumor conditions), targeted destruction of the tumor should be highly achievable regardless of the degree of vascularization, with tissue damage isolated to the malignant tissue at vascular sites and cellular sites.

### Drawing Legend

| No. | Description |
|---|---|
| 1 | quantum dot source, including tail |
| 1a | Quantum dot |
| 1b | Photosensitizer |
| 1c | Aptamer |
| 1d | Tail on quantum dot |
| 2 | Dextran iron oxide hybrid nanoparticle |
| 2a | amino dextran coated-iron oxide hybrid nanoparticles (IONP's) |
| 2b | N-BOC-AOA |
| 2c | ¹⁸F-FDG |
| 2d | PEG |
| 3 | 2,2′-Azobis(2-(2-imidazolin-2-yl)propane) dihydrochloride (AIPH) |
| 4 | D-Sorbitol-PEDOT:PSS |
| 5 | glycidyl methacrylate modified hyaluronic acid (HA-GMA) |
| 5a | Hyaluronic acid (HA) |
| 5b | glycidyl methacrylate (GM) |
| 5c | HA-GMA/GMHA |
| 6 | N-isopropyl acrylamide (NIPAM). |
| 7 | core region |
| 7a | CoFe2O4-BaTiO3 core-shell magnetoelectric nanobeads |
| 8 | Immunological factors |
| 8a | N-formylmethionine-leucyl-phenylalanine (fMLF) |
| 8b | neoleukin-2/15 (Neo-2/15, or some other IL-2-like derivative |
| 9 | heterogenous intermediate layer |
| 10 | assembly outer layer |
| 100 | Nanoparticle assembly |
| 110 | Nanoparticles administered to a patient |
| 112 | Nanoparticles adhere to penetrate a tumor cell |
| 114 | AC magnetic field applied to the nanoparticle |
| 116 | IONP's vibrate due to magnetostriction |
| 118 | Vibration effects from magnetostriction |
| 120 | Piezoelectric effect generates electric charges in 7a nanobeads |
| 122 | Electric charge from 7a nanobeads is transmitted through electroconductive D |
| | polymers. |
| 124 | Electric charge activates QD's to cause fluorescence |
| 126 | Fluorescing QD's activate photosensitizer molecules |
| 128 | Reactive oxygen species generated |
| 130 | O• (reactive oxygen species) damages cell, inducing apoptosis |
| 139 | Heat effects due to magnetostriction |
| 140 | Heat from vibrating IONP's generates free radicals from AIPH in low O₂ environment. |
| 142 | Free radicals from AIPH induce apoptosis |
| 150 | Immunological factors (F) released |
| 152 | Immunological factors (MP's, CD4, CD8 cells) attack and kill cancer cells. |
| 160 | PEDOT:PSS degrades from heat facilitating clearance of the nanoparticles. |
| 170 | FDG |
| 210 | Preparation of nanobeads 7a |
| 212 | Incorporation of NIPAM/HA-GMA hydrogel into D-Sorbitol-PEDOT:PSS |
| 214 | Nanobeads coated with D-Sorbitol-PEDOT:PSS/NIPAM/HA-GMA hydrogel (9) |
| 216 | AIPH added to hydrogel |
| 217 | Incorporate STING agonists into hydrogel |
| 218 | Prepare IONP's (2) |
| 220 | Prepare aptamers (1c) |
| 222 | Prepare Quantum Dot Source (1) |
| 224 | Final assembly |

### Abbreviation Listing

| Abbreviation | Description |
|---|---|
| AIPH | 2,2′-Azobis(2-(2-imidazolin-2-yl)propane) dihydrochloride |
| AOA | aminooxyacetic acid (2b) |
| BTO | BaTiO₃ |
| CFO | CoFe₂O₄ |
| D-Sorbitol- | sorbitol-treated poly(3,4-ethylenedioxythiophene) |
| PEDOT:PSS | polystyrene sulfonate |
| FDG | 2-¹⁸Fluoro-2-deoxyglucose |
| GM | Glycidyl methacrylate |
| HA-GMA | glycidyl methacrylate modified hyaluronic acid |
| IONP | Iron oxide nanoparticle |
| NIPAM | N-isopropyl acrylamide |
| PET-CT | Positron emission tomography-computer tomography |
| QD | Quantum dot |
| STING | Stimulator of interferon genes |

### REFERENCES

1. Lee, J.; Sharei, A.; Sim, W. Y.; Adamo, A.; Langer, R.; Jensen, K. F.; Bawendi, M. G., Nonendocytic delivery of functional engineered nanoparticles into the cytoplasm of live cells using a novel, high-throughput microfluidic device. Nano Lett 2012, 12, 6322-7, DOI: 10.1021/nl303421h.
2. He, H.; Zhang, L.; Guan, X.; Cheng, H.; Liu, X.; Yu, S.; Wei, J.; Ouyang, J., Biocompatible Conductive Polymers with High Conductivity and High Stretchability. ACS Applied Materials & Interfaces 2019, 11, 26185-26193, DOI: 10.1021/acsami.9b07325.
3. Onorato, A.; Invernale, M. A.; Berghorn, I. D.; Pavlik, C.; Sotzing, G. A.; Smith, M. B., Enhanced conductivity in sorbitol-treated PEDOT-PSS. Observation of an in situ cyclodehydration reaction. Synthetic Metals 2010, 160, 2284-2289, DOI:
   https://doi.org/10.1016/j.synthmet.2010.08.021.
4. Kim, C.; Hong, J.; Park, J. W., Synthesis and Thermoelectric Properties of Selenium Nanoparticles Coated with PEDOT: PSS. Polymers (Basel) 2019, 11, DOI: 10.3390/polym11061052.
5. Sun, X.; Liu, D.; Xu, X.; Shen, Y.; Huang, Y.; Zeng, Z.; Xia, M.; Zhao, C., NIR-triggered thermo-responsive biodegradable hydrogel with combination of photothermal and thermodynamic therapy for hypoxic tumor. Asian J Pharm Sci 2020, 15, 713-727, DOI: 10.1016/j.ajps.2019.11.007.
6. Amino functionalized IONP are commercially available from Nanocs, Inc. New York, NY http://www.nanocs.net/
7. Fina Biosciences Amino-dextran supplier. https://www.finabio.net/product/amino-dextrans/(Accessed: Oct. 24, 2021).
8. De Simone, M.; Panetta, D.; Bramanti, E.; Giordano, C.; Salvatici, M. C.; Gherardini, L.; Menciassi, A.; Burchielli, S.; Cinti, C.; Salvadori, P. A., Magnetically driven nanoparticles: 18 FDG-radiolabelling and positron emission tomography biodistribution study. Contrast media & molecular imaging 2016, 11 6, 561-571,
9. Di, L., Strategic approaches to optimizing peptide ADME properties. AAPS J 2015, 17, 134-143, DOI: 10.1208/s12248-014-9687-3.
10. Thornburg, J. M.; Nelson, K. K.; Clem, B. F.; Lane, A. N.; Arumugam, S.; Simmons, A.; Eaton, J. W.; Telang, S.; Chesney, J., Targeting aspartate aminotransferase in breast cancer. Breast Cancer Research 2008, 10, R84, DOI: 10.1186/bcr2154.
11. Olabi, A. G.; Grunwald, A., Design and application of magnetostrictive materials. Materials & Design 2008, 29, 469-483, DOI: https://doi.org/10.1016/.matdes.2006.12.016.
12. Dembowski, S. K.; Bowser, M. T., CE-SELEX: Rapid Aptamer Selection Using Capillary Electrophoresis. University of Minnesota, M., Minnesota, Ed.: Sciex white paper, 2016.
13. Quantum dots, nanocomposite materials with quantum dots, devices with quantum dots, and related fabrication methods. U.S. Pat. No. 7,005,669B1.
14. Rao, B. N.; Kaviraj, P.; Vaibavi, S. R.; Kumar, A.; Bajpai, S. K.; Arockiarajan, A., Investigation of magnetoelectric properties and biocompatibility of CoFe2O4-BaTiO3 core-shell nanoparticles for biomedical applications. Journal of Applied Physics 2017, 122, 164102, DOI: 10.1063/1.4993831.
15. Genchi, G.; Sinicropi, M. S.; Lauria, G.; Carocci, A.; Catalano, A., The Effects of Cadmium Toxicity. Int J Environ Res Public Health 2020, 17, DOI: 10.3390/ijerph17113782.
16. Base Pair Technologies, I. Aptamer Advantages. https://www.basepairbio.com/aptamer-advantages/(Accessed: Oct. 24, 2021).
17. Wu, Q.; Cao, F.; Kong, L.; Yang, X., InP quantum dots-based electroluminescent devices*. Chinese Physics B 2019, 28, 118103, DOI: 10.1088/1674-1056/ab4cdb.
18. Yang, J.; Xie, R.; Feng, L.; Liu, B.; Lv, R.; Li, C.; Gai, S.; He, F.; Yang, P.; Lin, J., Hyperthermia and Controllable Free Radical Coenhanced Synergistic Therapy in Hypoxia Enabled by Near-Infrared-II Light Irradiation. ACS Nano 2019, 13, 13144-13160, DOI: 10.1021/acsnano.9b05985.
19. Dobson, J.; Wilson, M., Sensitization of oral bacteria in biofilms to killing by light from a low-power laser. Archives of Oral Biology 1992, 37, 883-887, DOI: https://doi.org/10.1016/0003-9969 (92) 90058-G.
20. Wilson, M.; Dobson, J.; Sarkar, S., Sensitization of periodontopathogenic bacteria to killing by light from a low-power laser. Oral Microbiology and Immunology 1993, 8, 182-187, DOI: https://doi.org/10.1111/j.1399-302X.1993.tb00663.x.
21. HASAN, T. Photosensitzer conjugates for targeting intracellular pathogens. US2002/0122805 A1, 2002.
22. Awuah, S. G.; You, Y., Boron dipyrromethene (BODIPY)-based photosensitizers for photodynamic therapy. RSC Advances 2012, 2, 11169-11183, DOI: 10.1039/C2RA21404K.
23. Jiang, Y.; Weiss, E. A., Colloidal Quantum Dots as Photocatalysts for Triplet Excited State Reactions of Organic Molecules. Journal of the American Chemical Society 2020, 142, 15219-15229, DOI: 10.1021/jacs.0c07421.
24. Gallagher, W. M.; Allen, L. T.; O'Shea, C.; Kenna, T.; Hall, M.; Gorman, A.; Killoran, J.; O'Shea, D. F., A potent nonporphyrin class of photodynamic therapeutic agent: cellular localisation, cytotoxic potential and influence of hypoxia. British Journal of Cancer 2005, 92, 1702-1710, DOI: 10.1038/sj.bjc.6602527.
25. Byrne, A. T.; O'Connor, A. E.; Hall, M.; Murtagh, J.; O'Neill, K.; Curran, K. M.; Mongrain, K.; Rousseau, J. A.; Lecomte, R.; McGee, S.; Callanan, J. J.; O'Shea, D. F.; Gallagher, W. M., Vascular-targeted photodynamic therapy with BF2-chelated Tetraaryl-Azadipyrromethene agents: a multi-modality molecular imaging approach to therapeutic assessment. British Journal of Cancer 2009, 101, 1565-1573, DOI: 10.1038/sj.bjc.6605247.
26. Wilson, W. R.; Hay, M. P., Targeting hypoxia in cancer therapy. Nature Reviews Cancer 2011, 11, 393-410, DOI: 10.1038/nrc3064.
27. Gao, D.; Shi, Y.; Ni, J.; Chen, S.; Wang, Y.; Zhao, B.; Song, M.; Guo, X.; Ren, X.; Zhang, X.; Tian, Z.; Yang, Z., NIR/MRI-Guided Oxygen-Independent Carrier-Free Anti-Tumor Nano-Theranostics. Small 2021, n/a, 2106000, DOI: https://doi.org/10.1002/smll.202106000.
28. Zhang, L.; Fan, Y.; Yang, Z.; Yang, M.; Wong, C. Y., NIR-II-driven and glutathione depletion-enhanced hypoxia-irrelevant free radical nanogenerator for combined cancer therapy. J Nanobiotechnology 2021, 19, 265, DOI: 10.1186/s12951-021-01003-2.
29. Xia, R.; Zheng, X.; Hu, X.; Liu, S.; Xie, Z., Photothermal-Controlled Generation of Alkyl Radical from Organic Nanoparticles for Tumor Treatment. ACS Applied Materials & Interfaces 2019, 11, 5782-5790, DOI: 10.1021/acsami.8b18953.
30. Werber, J.; Wang, Y. J.; Milligan, M.; Li, X.; Ji, J. A., Analysis of 2,2′-azobis (2-amidinopropane) dihydrochloride degradation and hydrolysis in aqueous solutions. J Pharm Sci 2011, 100, 3307-3315, DOI: 10.1002/jps.22578.
31. Silva, D. A.; Yu, S.; Ulge, U. Y.; Spangler, J. B.; Jude, K. M.; Labão-Almeida, C.; Ali, L. R.; Quijano-Rubio, A.; Ruterbusch, M.; Leung, I.; Biary, T.; Crowley, S. J.; Marcos, E.; Walkey, C. D.; Weitzner, B. D.; Pardo-Avila, F.; Castellanos, J.; Carter, L.; Stewart, L.; Riddell, S. R.; Pepper, M.; Bernardes, G. J. L.; Dougan, M.; Garcia, K. C.; Baker, D., De novo design of potent and selective mimics of IL-2 and IL-15. Nature 2019, 565, 186-191, DOI: 10.1038/s41586-018-0830-7.
32. Neoleukin Therapeutics, I. NL-201: CD25-Independent IL-2/IL-15 Agonist Immunotherapy. https://www.neoleukin.com/programs/(Accessed: Oct. 24, 2021).
33. MANZANO, D. A. S.; RUBIO, A. Q.; GUTIERREZ, J. R. V.; Linsky, T.; NELSON, J. Interleukin-2 receptor βeta (il-2rβ) binding polypeptides WO2021188374A2, 2021.
34. Amouzegar, A.; Chelvanambi, M.; Filderman, J. N.; Storkus, W. J.; Luke, J. J., STING Agonists as Cancer Therapeutics. Cancers 2021, 13, 2695, DOI:
   doi: 10.3390/cancers13112695.
35. SASO, C. Cyclic dinucleotide STING agonists for cancer treatment. WO2018/118665 A1, 2018.
36. RYOSUKE, H. STING agonistic compound. US20210363166 A1, 2021.
37. Onyedibe, K. I.; Wang, M.; Sintim, H. O., ENPP1, an Old Enzyme with New Functions, and Small Molecule Inhibitors-A STING in the Tale of ENPP1. Molecules 2019, 24, 4192, DOI: doi: 10.3390/molecules24224192.
38. LI, L. ENPP1 inhibitors and methods of modulating immune response. WO2020/160333 A1, 2020.
39. COGAN, D. A. Heterocyclic inhibitors of ENPP1. WO2021/257614 A1, 2021.
40. Ding, C.; Song, Z.; Shen, A.; Chen, T.; Zhang, A., Small molecules targeting the innate immune cGAS-STING-TBK1 signaling pathway. Acta Pharmaceutica Sinica B 2020, 10, 2272-2298, DOI: https://doi.org/10.1016/j.apsb.2020.03.001.
41. Gao, M.; He, Y.; Tang, H.; Chen, X.; Liu, S.; Tao, Y., cGAS/STING: novel perspectives of the classic pathway. Molecular Biomedicine 2020, 1, 7, DOI: 10.1186/s43556-020-00006-z.
42. Wu, Y. Y., A novel DNA aptamer targeting lung cancer stem cells exerts a therapeutic effect by binding and neutralizing Annexin A2. Mol Ther Nucl Acids 2022, 27, 956-968, DOI: https://doi.org/10.1016/j.omtn.2022.01.012.
43. Kumar Kulabhusan, P.; Hussain, B.; Yüce, M., Current Perspectives on Aptamers as Diagnostic Tools and Therapeutic Agents. Pharmaceutics 2020, 12, 646, DOI: https://doi.org/10.3390/pharmaceutics 12070646.
44. Kim, Y.; Wu, Q.; Hamerlik, P.; Hitomi, M.; Sloan, A. E.; Barnett, G. H.; Weil, R. J.; Leahy, P.; Hjelmeland, A. B.; Rich, J. N., Aptamer Identification of Brain Tumor-Initiating Cells. Cancer Research 2013, 73, 4923-4936, DOI: 10.1158/0008-5472.Can-12-4556.
45. Biaoxue, R.; Xiguang, C.; Shuanying, Y., Annexin A1 in Malignant Tumors: Current Opinions and Controversies. The International Journal of Biological Markers 2014, 29, e8-e20, DOI: 10.5301/jbm.5000046.
46. Veronese, F. M.; Mero, A., The Impact of PEGylation on Biological Therapies. BioDrugs 2008, 22, 315-329, DOI: 10.2165/00063030-200822050-00004.
47. Specht, L., 2-[18F] Fluoro-2-Deoxyglucose Positron-Emission Tomography in Staging, Response Evaluation, and Treatment Planning of Lymphomas. Seminars in Radiation Oncology 2007, 17, 190-197, DOI: https://doi.org/10.1016/Lsemradonc.2007.02.005.
48. Wilson, D. R.; Sen, R.; Sunshine, J. C.; Pardoll, D. M.; Green, J. J.; Kim, Y. J., Biodegradable STING agonist nanoparticles for enhanced cancer immunotherapy. Nanomedicine 2018, 14, 237-246, DOI: 10.1016/j.nano.2017.10.013.
49. Shae, D.; Becker, K. W.; Christov, P.; Yun, D. S.; Lytton-Jean, A. K. R.; Sevimli, S.; Ascano, M.; Kelley, M.; Johnson, D. B.; Balko, J. M.; Wilson, J. T., Endosomolytic polymersomes increase the activity of cyclic dinucleotide STING agonists to enhance cancer immunotherapy. Nature Nanotechnology 2019, 14, 269-278, DOI: 10.1038/s41565-018-0342-5.
50. Liu, H.; Wang, C.; Li, C.; Qin, Y.; Wang, Z.; Yang, F.; Li, Z.; Wang, J., A functional chitosan-based hydrogel as a wound dressing and drug delivery system in the treatment of wound healing. RSC Advances 2018, 8, 7533-7549, DOI: 10.1039/C7RA13510F.
51. De Simone, M.; Panetta, D.; Bramanti, E.; Giordano, C.; Salvatici, M. C.; Gherardini, L.; Menciassi, A.; Burchielli, S.; Cinti, C.; Salvadori, P. A., Magnetically driven nanoparticles: (18) FDG-radiolabelling and positron emission tomography biodistribution study. Contrast Media Mol Imaging 2016, 11, 561-571, DOI: 10.1002/cmmi.1718.
52. Labiadh, H.; Hidouri, S., ZnS quantum dots and their derivatives: Overview on identity, synthesis and challenge into surface modifications for restricted applications. Journal of King Saud University-Science 2017, 29, 444-450, DOI: https://doi.org/10.1016/j.jksus.2016.12.001.
53. Chen, O.; Riedemann, L.; Etoc, F.; Herrmann, H.; Coppey, M.; Barch, M.; Farrar, C. T.; Zhao, J.; Bruns, O. T.; Wei, H.; Guo, P.; Cui, J.; Jensen, R.; Chen, Y.; Harris, D. K.; Cordero, J. M.; Wang, Z.; Jasanoff, A.; Fukumura, D.; Reimer, R.; Dahan, M.; Jain, R. K.; Bawendi, M. G., Magneto-fluorescent core-shell supernanoparticles. Nature Communications 2014, 5, 5093, DOI: 10.1038/ncomms6093.

1.-10. (canceled)

11. A nanoparticle assembly for the treatment and visualization of a cancer, comprising:
a. an assembly core (7) comprising a plurality of nanobeads (7a), where each nanobead has a nested core-shell configuration wherein the core comprises CoFe₂O₄ and the shell comprises BaTiO₃, and wherein the nanobeads are magnetoelectric;
b. wherein the assembly core is bounded by an intermediate copolymer layer (9) comprising D-Sorbitol-PEDOT: PSS (4), a biocompatible polymer filler (5) that adjusts the lower critical solution temperature (LCST) of N-isopropyl acrylamide (6), an azobis compound (3) that generates free radicals thermolytically, and aminooxyacetic group-conjugated dextran-iron oxide hybrid nanoparticles (2a); and
c. wherein the assembly comprises an outer layer (10) comprising iron oxide hybrid nanoparticles (2) comprising an iron oxide nanoparticle core with a tail comprising one or more aminooxyacetate linkers (2b) and optionally one or more 2-¹⁸Fluoro-2-deoxyglucose (¹⁸F-FDG) subunits (2c); wherein the nanoparticles (2) further comprise a polyethylene glycol (2d) moiety and an aptamer conjugated thereto.

12. The assembly of claim 11, wherein the azobis compound is selected from 2,2′-Azobis(2-(2-imidazolin-2-yl) propane) dihydrochloride (AIPH); dimethyl 2,2′-azobis (2-methylpropionate) (AIBME); and 2,2′-Azobis(2-amidinopropane) dihydrochloride (AAPH).

13.-17. (canceled)

18. A method of killing cancer cells in a patient suffering from a cancerous tumor, comprising:
a. the administration of the nanoparticle assembly of claim 11 to a patient suffering from a cancer or malignant tumor, waiting a period of time following administration to allow the assemblies to enter into or attach to the surface of cancer cells, and subjecting an area affected by cancer to an alternating current (AC) magnetic field; and
b. wherein the AC magnetic field causes magnetostriction in the nanobeads (7a), and wherein magnetic induction generates heat causing the polymer material (4) to deform and warm up, thereby generating free radicals from an azobis free radical precursor (3), and wherein the free radicals induce cellular damage and apoptosis in cancer cells.

19.-21. (canceled)

22. The method of claim 18, wherein the nanoassembly is guided to the site of a tumor with a DC magnetic field with or without computed tomographic scanning visualization.

23. The nanoparticle assembly of claim 11, wherein the nanobeads (7a) further comprise a nested core-shell configuration wherein the core comprises CoFe₂O₄ (CFO) and the shell comprises BaTiO₃ (BTO).

24. The nanoparticle assembly of claim 11, wherein the biocompatible polymer filler is glycidyl methacrylate/glycidyl methacrylate hyaluronic acid (HA-GMA/GMHA) (5).

25. A method of generating free radicals within or adjacent to cancer cells in a patient suffering from a cancerous tumor, comprising:
a. the administration of the nanoparticle assembly of claim 11 to a patient suffering from a cancer or malignant tumor, waiting a period of time following administration to allow the assemblies to enter into or attach to the surface of cancer cells, and subjecting an area affected by cancer to an alternating current (AC) magnetic field; and
b. wherein the AC magnetic field causes magnetostriction in the nanobeads (7a) in the nanoparticle assembly, and wherein magnetic induction generates heat causing the polymer material (4) to deform and warm up with sufficient heat to generate free radicals from the azobis free radical precursor (3).
